(19) United States
(12) Patent Application Publication
(10) Pub. No.: US 2014/0343014 A1
(43) Pub. Date: Nov. 20, 2014

(54) COMBINATION OF A CERTAIN DPP-4 INHIBITOR AND VOGLIBOSE

(71) Applicants: Thomas KLEIN, Radolfzell (DE); Michael MARK, Biberach an der Riss (DE)

(72) Inventors: Thomas KLEIN, Radolfzell (DE); Michael MARK, Biberach an der Riss (DE)

(73) Assignee: BOEHRINGER INGELHEIM INTERNATIONAL GMBH, Ingelheim am Rhein (DE)

(21) Appl. No.: 14/279,683

(22) Filed: May 16, 2014

(30) Foreign Application Priority Data
May 17, 2013 (EP) 13168375.7

(51) Int. Cl. A61K 31/522; A61K 45/06; A61K 31/133

(52) U.S. Cl. CPC A61K 31/522; A61K 31/133; A61K 45/06
USPC 514/61; 514/263.21

(57) ABSTRACT

The present invention relates to the use of a combination of a certain DPP-4 inhibitor and an alpha-glucosidase inhibitor for use in therapy, e.g. for use in treating and/or preventing a metabolic disease such as type 2 diabetes mellitus and/or conditions related thereto. The invention also relates to the use of such combination for improving body weight control, reducing body weight, inducing satiety, inhibiting gastric emptying and/or reducing food intake, in a patient in need thereof.

### FIELD OF THE INVENTION

The present invention relates to a combination of a certain DPP-4 inhibitor (preferably linagliptin) and an alpha-glucosidase inhibitor (e.g. voglibose, miglitol or acarbose, such as in low dose form; preferably voglibose), such as e.g. for use in therapy, e.g. for use in treating and/or preventing a metabolic disease, particularly diabetes, especially type 2 diabetes mellitus (such as e.g. including high postprandial glycemia, and/or conditions related thereto, e.g. diabetic complications, such as e.g. micro- or macrovascular diseases such as cardio- or cerebrovascular diseases, nephropathy, retinopathy or neuropathy) and/or for improving body weight control, reducing body weight, inducing satiety, inhibiting gastric emptying and/or reducing food intake, in a patient in need thereof, such as e.g. in a diabetes patient (optionally having or being at-risk of micro- or macrovascular disease or complication), such as preferably in a type 2 diabetes patient having or being at-risk of obesity or overweight, and/or having or being at-risk of hepatic or renal impairment (such as e.g. of mild, moderate, severe or ESRD stage) and/or, optionally, with indication/recommendation on dose reduction of an alpha-glucosidase inhibitor to be used (or such as e.g. in an elderly and/or renally impaired subject), wherein, optionally, the alpha-glucosidase inhibitor/blocker may be present or may be used in reduced amount and/or less frequent (daily) dosing (such as e.g. voglibose in low dose such as 0.2-0.3 mg, each one, two or three times a day; acarbose in low dose such as 25-50 mg (or up to 100 mg), each one, two or three times a day; or miglitol in low dose such as 25-50 mg (or up to 100 mg), each one, two or three times a day) when co-administered with the DPP-4 inhibitor (preferably linagliptin).

The present invention further relates to the use of a certain DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose)) to specifically modify and/or reduce the intake of food by a subject, particularly wherein said food is rich in fat and/or in carbohydrate (e.g. high caloric, palatable, sweet and/or fatty food), for example such food which has a high glycemic index and/or wherein the amount of mono- and/or di-saccharides constitute a large portion of the total amount of carbohydrate and/or wherein a large portion of the total amount of energy stems from fat.

### BACKGROUND OF THE INVENTION

Lifestyle in many parts of the world today is characterized by an enormous meal and “between-meal” intake of calories (e.g. from solid food and snacks as well as drinkable calories), often associated with or provoked by multiple temptations of an “adipogenic”, sedentary and calorie-laden environment and shortage of physical exercise.

This lifestyle is often referred to as “western world lifestyle”, and it is generally regarded as unhealthy. Our food earlier consisted of an average of 10% protein, 30% fat and 60 carbohydrates; the carbohydrates mostly in the form of slowly absorbed carbohydrates. The food and especially the between-meal snack consumed today often has a much higher amount of quickly absorbed carbohydrates and fat. The amount of quickly absorbed carbohydrates may be measured as the glycemic index or as the fraction of mono- and di-saccharide of the total amount of carbohydrates. The excess intake of quickly absorbed carbohydrates and/or high fat leads to reduced feelings of hunger and to increased stress. Also, some human beings have cravings for sweet and/or fat food, sometimes enhanced by stress or premenstrual tension, or they may have psychological problems manifested as binge eating or compulsive eating habits.

As a consequence of this western world lifestyle, eating behavior, food or taste preference and/or the psychological disorders described above there is a general excessive intake of food, especially of highly caloric unhealthy food, like sodas, juice, chocolate milk, sweetened coffee, candy, chocolate, cake, biscuits, crackers, french fries, burgers, white bread with jam or jelly or honey, chips, sweet and fat cereals.

Therefore, in addition or in alternative to conventional weight management algorithms like diet, exercise, behavioral or lifestyle modification or—especially in case of extremely obese subjects—bariatric (weight-loss) surgery, there remains a need for further options for weight management by pharmaceutical or pharmacotherapeutic interventions, such as for managing, treating or preventing weight gain, overweight, obesity or other (metabolic) diseases or disorders associated or related therewith, for promoting weight loss, for controlling body weight, for changing eating behavior, for regulating food or taste preference and/or for maintaining or adjusting adequate balance of healthy and unhealthy food consumption e.g. to meet individual's established nutritional requirements and a healthy nutritional lifestyle.

In the treatment of type 2 diabetes, alpha-glucosidase inhibitors (AGIs; including acarbose, miglitol, voglibose) are recommend by guidelines for glucose control in type 2 diabetes. AGIs delay the absorption of carbohydrates by the gut, by inhibiting alpha-glucosidase in the small intestine, and thus have an effect on lowering postprandial blood glucose and insulin levels.

### SUMMARY OF THE INVENTION

The present invention relates to a combination of a certain DPP-4 inhibitor (preferably linagliptin) and an alpha-glucosidase inhibitor/blocker (e.g. voglibose, miglitol or acarbose, preferably voglibose), such as e.g. for use in therapy (e.g. for improving glycemic and/or body weight control, reducing body weight, inducing satiety, inhibiting gastric emptying and/or reducing food intake, and/or for treating and/or preventing a metabolic disease, particularly diabetes, especially type 2 diabetes mellitus, and/or conditions related thereto, e.g. diabetic complications, such as e.g. micro- or macrovascular diseases such as cardio- or cerebrovascular diseases, nephropathy, retinopathy or neuropathy) in a patient in need thereof (e.g. diabetes patients optionally with or without obesity, such as e.g. those patients having or being at-risk of renal impairment e.g. with indication on dose reduction of an alpha-glucosidase inhibitor/blocker to be used, and/or having or being at-risk of micro- or macrovascular disease or complication), optionally in combination with one or more other active agents; such as e.g. wherein the alpha-glucosidase inhibitor/blocker is present or is used in reduced amount and/or less frequent (daily) dosing (such as e.g. voglibose in low dose such as 0.2 mg, 1-3 times a day; acarbose in low dose such as 25-50 mg (or up to 100 mg), each 1-3 times a day; or miglitol in low dose such as 25-50 mg (or up to 100 mg), each 1-3 times a day) when co-administered with the DPP-4 inhibitor (preferably linagliptin).

Furthermore, the present invention relates to a certain DPP-4 inhibitor (preferably linagliptin, optionally in combination with one or more other active agents) for use in preventing, protecting against, reducing (e.g. the likelihood or incidence of) or minimizing the side effects associated with the therapeutic use of alpha-glucosidase inhibitors/blockers (e.g. voglibose, miglitol or acarbose), such as gastrointestinal adverse effects (e.g. dyspepsia, flatulence or diarrhea, or nausea or vomiting).

Accordingly, in certain particular embodiments the present invention relates to the combination or combined use or combined administration (such as e.g. simultaneously, sequentially or separately) of a certain DPP-4 inhibitor (preferably linagliptin) and an alpha-glucosidase inhibitor (e.g. voglibose, miglitol or acarbose, such as in low dose form; preferably voglibose).

The present invention further relates to the use of a certain DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose) to specifically modify and/or reduce the intake of food by a subject, particularly wherein said food is rich in fat and/or in carbohydrate (e.g. high caloric, palatable, sweet and/or fatty food), for example such food which has a high glycemic index and/or wherein the amount of mono- and/or di-saccharides constitute a large portion of the total amount of carbohydrate and/or wherein a large portion of the total amount of energy stems from fat.

Further, the present invention relates to a principle for combined weight and pre-diabetes/diabetes management (such as e.g. for appetite suppression, change in diet preference, body weight loss and/or body weight control, e.g. beyond glycemic control) such as in obese (e.g. human) subjects by using a certain DPP-4 inhibitor (preferably linagliptin, optionally in combination with one or more other active agents, such as e.g. with a GLP-1 receptor agonist, such as a short acting GLP-1, GLP-1 mimetic or GLP-1 analogue, such as exenatide or native GLP-1, e.g. for appetite suppression, change in diet preference and/or body weight loss, and/or with an α-glucosidase inhibitor, such as voglibose, e.g. for body weight control).

The present invention further relates to the use of a certain DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose) for reducing the intake of high caloric (palatable, rich in fat and/or carbohydrate) food and/or for regulating or shifting food or taste preference or choice of food away from high caloric (palatable, rich in fat and/or carbohydrate) food intake, particularly with decreasing the amount of unhealthy and/or high caloric (e.g. palatable, rich in fat and/or carbohydrate) food intake and, optionally, with increasing (e.g. relatively to the amount of unhealthy and/or high caloric food intake) the amount of healthy and/or low caloric food intake.

Accordingly, the present invention relates to the use of a certain DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose) for altering eating attitudes and shifting eating and/or taste preference towards healthy food.

Accordingly, the present invention relates to the use of a certain DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose) for altering eating attitudes and shifting eating and/or taste preference away from unhealthy food.

Further, the present invention relates to the use of a certain DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose) for reducing, facilitating a reduction in, or preventing an increase in caloric intake, excessive food energy intake, body weight, total body fat mass, total body fat percentage, visceral fat and/or over eating.

In an embodiment, the subject described herein is overweight or obese, e.g. with or without risk factors for or comorbidities such as diabetes mellitus, dyslipidemia, hypertension and/or metabolic syndrome.

In particular, the subject described herein is overweight or obese, e.g. with or without diabetes.

In another embodiment, the subject described herein is a subject with an abnormal or excessive intake of food.

In another embodiment, the subject described herein is a subject having diabetes (e.g. type 1 or type 2 diabetes or LADA, particularly type 2 diabetes), e.g. with or without obesity or overweight.

In particular, the subject within this invention may be a human, e.g. a human child, a human adolescent or a human adult.

In an embodiment, the combination of a certain DPP-4 inhibitor (preferably linagliptin) and an alpha-glucosidase inhibitor/blocker (e.g. voglibose, miglitol or acarbose, preferably voglibose) may be particularly useful in subjects who exhibit a reluctance to avoid high-carbohydrate diet.

In an embodiment, the combination of a certain DPP-4 inhibitor (preferably linagliptin) and an alpha-glucosidase inhibitor/blocker (e.g. voglibose, miglitol or acarbose, preferably voglibose) may be particularly useful in an Asian population (e.g. East Asia, South Asia or Southeast Asia) and/or following an eastern diet with higher carbohydrate content.

In an embodiment, the combination of a certain DPP-4 inhibitor (preferably linagliptin) and an alpha-glucosidase inhibitor/blocker (e.g. voglibose, miglitol or acarbose, preferably voglibose) may be particularly useful for an Asian population (e.g. East Asia, South Asia or Southeast Asia) and/or for reducing, treating, preventing or managing the negative (metabolic) impact of unhealthy western diet.

In an embodiment, the combination of a certain DPP-4 inhibitor (preferably linagliptin) and an alpha-glucosidase inhibitor/blocker (e.g. voglibose, miglitol or acarbose, preferably voglibose) may be particularly useful for an Asian population (e.g. East Asia, South Asia or Southeast Asia) and/or for treating diabetes and/or obesity/overweight (such as particularly “diabesity”) and/or for changing (unhealthy) eating behavior, for regulating or changing (unhealthy) food or taste preference (towards healthy diet) and/or for maintaining or adjusting adequate balance of healthy and unhealthy food consumption e.g. to meet individual's established nutritional requirements and a healthy nutritional lifestyle.

For example, unhealthy food in the meaning of this invention may be food from one or more of the groups A) to D):

A) The glycemic index is above 60%
B) The glycemic index is above 40% and wherein more than 30% of the total amount of energy stems from fat
C) The amount of mono- and/or di-saccharides constitute more than 25% of total carbohydrate content
D) The amount of mono- and/or di-saccharides constitute more than 25% of total carbohydrate content and wherein more than 30% of the total amount of energy stems from fat.

For example, healthy food in the meaning of this invention may be food from one or more of the groups E) to H):

E) The glycemic index is below 60%
F) The glycemic index is below 40% and wherein less than 30% of the total amount of energy stems from fat
G) The amount of mono- and/or di-saccharides constitute together constitute less than 25% of the total carbohydrate content
H) The amount of mono- and/or di-saccharides constitute less than 25% of the total carbohydrate content, and wherein less than 30% of the total amount of energy stems from fat

Further, the present invention relates to a DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose)) for use in the treatment and/or prevention of a metabolic disease such as described herein (such as e.g. type 2 diabetes mellitus and/or obesity) in a patient in need thereof (such as e.g. a patient having overweight, obesity and/or diabetes), particularly

in a patient who is further in need of reduction in, facilitation of a reduction in, or prevention of an increase in caloric intake, excessive food energy intake, body weight, total body fat mass, total body fat percentage, visceral fat and/or over eating, and/or
in a patient who is further in need of modification of and/or reduction in intake of food, particularly wherein said food is rich in fat and/or in carbohydrate (e.g. high caloric, palatable, sweet and/or fatty food), for example such food which has a high glycemic index and/or wherein the amount of mono- and/or di-saccharides constitute a large portion of the total amount of carbohydrate and/or wherein a large portion of the total amount of energy stems from fat, and/or
in a patient who is further in need of reduction in intake of high caloric (palatable, rich in fat and/or carbohydrate) food and/or of regulation or shift in food or taste preference or choice of food away from unhealthy food (e.g. as described herein) and towards healthy food (e.g. as described herein), such as in a patient in need of decrease in the amount of unhealthy and/or high caloric (e.g. palatable, rich in fat and/or carbohydrate) food intake and/or in need of increase in the amount of healthy and/or low caloric food intake.

Accordingly, in a particular embodiment, a preferred DPP-4 inhibitor within the meaning of this invention is linagliptin.

Pharmaceutical compositions or combinations for use in these therapies (treatments or preventions) comprising a certain DPP-4 inhibitor (preferably linagliptin) as defined herein optionally together with one or more other active agents are also contemplated.

Further, the present invention relates to a certain DPP-4 inhibitor (preferably linagliptin), optionally in combination with one, two or more further active agents, each as defined herein, for use in the therapies (treatments or preventions) as described herein.

Further, the present invention relates to the use of a certain DPP-4 inhibitor (preferably linagliptin), optionally in combination with one, two or more further active agents, each as defined herein, for preparing a pharmaceutical composition which is suitable for the treatment and/or prevention purposes of this invention.

Further, the present invention relates to a therapeutic (treatment or prevention) method as described herein, said method comprising administering an effective amount of a certain DPP-4 inhibitor (preferably linagliptin) and, optionally, one or more other active or therapeutic agents to the patient in need thereof, each as described herein.

Other aspects of the present invention become apparent to the skilled person from the foregoing and following remarks (including the examples and claims).

The aspects of the present invention, in particular the pharmaceutical compounds, compositions, combinations, methods and uses, refer to a certain DPP-4 inhibitor (preferably linagliptin), optionally in combination with one or more other active agents, such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), as defined hereinbefore and hereinafter.

### BRIEF DESCRIPTION OF THE DRAWINGS

FIG. 1 shows bidaily s.c. treatment of male Sprague Dawley rats (obesity model) with linagliptin and native GLP-1 leading to a significant reduction of body weight (BW) up to 8%. Data show mean values+SEM (standard error of the mean) of 10 animals.

FIG. 2 shows significant decrease in fat mass following application of the combination of linagliptin and native GLP-1 in these animals.

FIGS. 3a and 3b show that the Linagliptin/GLP-1 treated animals possess altered food preferences in reduction intake of high fat diet a) and switching to chow diet b).

### DETAILED DESCRIPTION OF THE INVENTION

The glycemic index is a measure of the ability of food to raise the blood glucose level. The glycemic index of a food can be determined by feeding a group consisting of at least 10 healthy people a portion of food containing 50 grams of digestible (available) carbohydrate and then measure the effect on their blood glucose levels during the following two hours. For each person, the area under their two-hour blood glucose response (glucose AUC) is measured. On another occasion, the same group of people consumes 50 g of glucose, and their two-hour blood glucose response is also measured. The glycemic index for the food is the AUC determined for the food divided by the AUC determined for glucose multiplied by 100% (calculated as the average for the group). Food with a high glycemic index contain rapidly digested carbohydrate, which produces a large rapid rise and fall in the level of blood glucose. In contrast, foods with a low glycemic index score contain slowly digested carbohydrate, which produces a gradual, relatively low rise in the level of blood glucose.

In the present context, mono-saccharides are intended to indicate a carbohydrate that cannot be hydrolysed to simpler carbohydrates. The most relevant mono-saccharides in food are glucose and fructose.

In the present context, di-saccharides are intended to indicate carbohydrates which can be hydrolysed into two mono-saccharides. The most relevant di-saccharides in food are sucrose, maltose and lactose.

The amount of mono- and/or di-saccharides in food may be analysed specifically by enzymatic, gas-liquid chromatography (GLC) or high performance liquid chromatography (HPLC) methods. Depending on the food matrix to be analyzed, extraction of the low molecular weight carbohydrates in aqueous ethanol, usually 80% (v/v), may be advisable before analysis. Relevant analysis methods are provided in e.g. Southgate, “Determination of food carbohydrates”, Elsevier, Science Publishers, Barkinggate, 1991; Greenfield, “Food composition data. Production, management and use”, Elsevier Appleid Science, London, 1992; and Department of Health, “Dietery sugars and human health, Her Majesty's Stationary Office, London, 1989.

In the present context, carbohydrates can be defined as in “Carbohydrates in human nutrition. (FAO Food and Nutrition Paper-66)”, Report of a Joint FAO/WHO Expert Consultation, Rome, 14-18 Apr. 1997, Report of a Joint FAO WHO Expert Consultation Rome, 14-18 Apr. 1997, namely as polyhydroxy aldehydes, ketones, alcohols, acids, their simple derivatives and their polymers having linkages of the acetal type.

In the present context, fat is intended to indicate mono-, di- and/or tri-carboxylic acid ester derived from glycerol and cholesterol, where the glycerols are the more important source of energy in the food of the two. The amount of fat in food may be determined as disclosed in FAO: Food energy—methods of analysis and conversion factors, Report of a Technical Workshop, Rome, 3-6 Dec. 2002.

In the present context, total carbohydrate content is intended to indicate the sum of carbohydrates present in the food. It is not measured as such, but rather calculated as the difference between the total weight of the food and the sum of the weights of the non-carbohydrate components [FAO: Food energy—methods of analysis and conversion factors, Report of a Technical Workshop, Rome, 3-6 Dec. 2002.

In the present context, food, unless otherwise stated, is intended to indicate food in any form, i.e. both liquid and solid food, as well as basic food and candy, snacks, etc.

In the present context, reducing intake of food is intended to indicate that the amount of food (measured by its energy content) eaten by a group consisting of one or more subjects being administered a certain DPP-4 inhibitor, optionally in combination with another active agent, is reduced compared to a similar control group not being administered a certain DPP-4 inhibitor, optionally in combination with another active agent, as provided in the present invention.

Similarly, increasing intake of food is intended to indicate that the amount of food (measured by its energy content) eaten by a group consisting of one or more subjects being administered a certain DPP-4 inhibitor, optionally in combination with another active agent, is increased compared to a similar control group not being administered a certain DPP-4 inhibitor, optionally in combination with another active agent, as provided in the present invention.

In the present context, abnormal or excessive intake of food is intended to indicate an intake with pathological consequences, such as obesity, or which can be ascribed to a psychological state connected with e.g. pregnancy or premenstrual tension, or to a psychological disease, such as binge eating or compulsory eating habits.

An effective amount of a compound as used herein means an amount sufficient to affect, e.g. cure, alleviate or partially arrest the manifestations (e.g. clinical manifestations) of a given state or condition, such as a disease or disorder, and its complications. An amount adequate to accomplish this is defined as “effective amount”. Effective amounts for each purpose will depend on the severity of the condition, disease or injury as well as the weight and general state of the subject and mode of administration, or the like. It will be understood that determining an appropriate dosage may be achieved using routine experimentation, by constructing a matrix of values and testing different points in the matrix, which is all within the ordinary skills of a trained physician or veterinary.

In the present context, treatment or treating mean the management and care of a patient or subject for the purpose of combating a condition, such as a disease or a disorder. The term is intended to include the full spectrum of treatments for a given condition from which the patient or subject is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent or delay the onset of the symptoms or complications.

Within the meaning of this invention, unless otherwise noted, the uses or methods according to the invention may relate either to medical, such as therapeutic and/or prophylactic, uses/methods or to non-medical, such as non-therapeutic and/or non-prophylactic, uses/methods (such as e.g. cosmetic uses or methods).

Accordingly, within the meaning of this invention, unless otherwise noted, the treatments and/or preventions according to the invention may relate either to treatments and/or preventions for medical, such as therapeutic and/or prophylactic, purposes or to treatments and/or preventions for non-medical, such as non-therapeutic and/or non-prophylactic, purposes (such as e.g. cosmetic or lifestyle purposes).

Thus, in one aspect, the present invention provides the use of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to modify or reduce the intake of specific types of food (e.g. unhealthy food) by a subject (e.g. a subject with overweight, obesity and/or diabetes) wherein the food has a high glycemic index and/or wherein the mono- and/or di-saccharides constitute a large proportion of the total amount of carbohydrates in said food and/or wherein a large proportion of the total amount of energy stems from fat in said food.

In a further aspect, the invention provides a method for decreasing the intake of food by a subject, wherein the food has a high glycemic index, said method comprising the administration of an effective amount of a certain DPP-4 inhibitor, optionally in combination with one or more other active agents (such as e.g. a GLP-1 receptor agonist, particularly a short acting GLP-1, GLP-1 mimetic or GLP-1 analogue, such as exenatide or native GLP-1), to said subject.

Particularly in this aspect, the glycemic index of the food may be above 60%, such as above 65%, such as above 70%, such as above 75%, such as above 80%, such as above 90%.

In a further aspect, the invention provides a method for decreasing the intake of food by a subject, wherein the mono- and/or di-saccharides constitute a large proportion of the total amount of carbohydrates in said food, said method comprising the administration of an effective amount of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to said subject.

Particularly in this aspect, the amount of mono- and/or di-saccharides together of total carbohydrate content may be above 25%, such as above 30%, such as above 40%, such as above 50%, such as above 60%, such as above 70%, such as above 80%, such as above 90%, or even 100%.

In an embodiment of this aspect, the mono-, di- and tri-saccharides together constitute more than 25% of the total amount of carbohydrates.

In a further aspect, the invention provides a method for decreasing the intake of food by a subject, wherein the food has a glycemic index above 40%, and wherein more than 30% of the total amount of energy stems from fat in said food, said method comprising administering an effective amount of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to said subject.

There is included in this aspect any combination of food with a glycemic index above 40%, such as above 45%, such as above 50%, such as above 55%, such as above 60%, such as above 65%, such as above 70%, such as above 75%, such as above 80%, such as above 90%, and wherein more than 30%, such as more than 35%, such as more than 40%, such as more than 50%, such as more than 60%, such as more than 70%, such as more than 80% of the total amount of energy stems from fat.

In a further aspect, the invention provides a method of decreasing the intake of food by a subject, wherein mono- and/or di-saccharides together constitute more than 25% of the total amount of carbohydrates and wherein more than 30% of the total amount of energy stems from fat in said food, said method comprising administering to said subject an effective amount of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

There is included in this aspect any combination of food wherein mono- and/or di-saccharides together constitute more than 25%, such as more than 30%, such as more than 40%, such as more than 45%, such as more than 50%, such as more than 70%, such as more than 80%, such as more than 90%, or even 100% of the total amount of carbohydrates, and wherein more than 30%, such as more than 35%, such as more than 40%, such as more than 50%, such as more than 60%, such as more than 70%, such as more than 80% of the total amount of energy stems from fat.

Further thus, in another aspect, the present invention provides the use of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to modify or increase the intake of specific types of food (e.g. healthy food) by a subject (e.g. a subject with overweight, obesity and/or diabetes) wherein the food has a low glycemic index and/or wherein the mono- and/or di-saccharides constitute a small proportion of the total amount of carbohydrates in said food and/or wherein a small proportion of the total amount of energy stems from fat in said food.

In a further aspect, the invention provides a method of increasing the intake of food by a subject, wherein the food has a low glycemic index, said method comprising the administration of an effective amount of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to said subject

Particularly in this aspect, the glycemic index of the food may be below 60%, such as below 50%, such as below 40%, such as below 35%, such as below 30%, such as below 20%, such as below 10%, such as below 5%.

In a further aspect, the invention provides a method of increasing the intake of food by a subject, wherein the mono- and/or di-saccharides constitute a small proportion of the total amount of carbohydrates in said food, said method comprising the administration of an effective amount of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to said subject

Particularly in this aspect, the amount of mono- and/or di-saccharides together of total carbohydrate content may be below 25%, such as below 20%, such as below 15%, such as below 10%, such as below 5%.

In an embodiment of this aspect, the mono-, di- and tri-saccharides together constitute less than 25% of the total amount of carbohydrates.

In a further aspect, the invention provides a method of increasing the intake of food by a subject, wherein the food has a glycemic index below 40%, and wherein less than 30% of the total amount of energy stems from fat in said food, said method comprising administering an effective amount of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to said subject.

There is included in this aspect any combination of food with a glycemic index below 40%, such as below 30%, such as below 20%, such as below 10%, such as below 5%, and wherein less than 30%, such as less than 20%, such as less than 10%, such as less than 5% of the total amount of energy stems from fat.

In a further aspect, the invention provides a method of increasing the intake of food by a subject, wherein mono- and/or di-saccharides together constitute less than 25% of the total amount of carbohydrates and wherein less than 30% of the total amount of energy stems from fat in said food, said method comprising administering to said subject an effective amount of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

There is included in this aspect any combination of food wherein mono- and/or di-saccharides together constitute less than 25%, such as less than 20%, such as less than 15%, such as less than 10%, such as less than 5% of the total amount of carbohydrates, and wherein less than 30%, such as less than 25%, such as less than 20%, such as less than 15%, such as less than 10%, such as less than 5% of the total amount of energy stems from fat.

In another aspect, the decrease in intake of food with a high glycemic index and/or food wherein mono- and/or di-saccharides constitute a large proportion of the total amount of carbohydrates and/or food wherein a large proportion of the total amount of energy stems from fat, as discussed above, is accompanied by an increase in the intake of food with a low glycemic index and/or of food wherein mono- and/or di-saccharides constitute as small proportion of the total amount of carbohydrates and/or food wherein a small proportion of the total amount of energy stems from fat, as discussed above.

Further thus, in another aspect, the present invention provides the use of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to regulate or shift food or taste preference or choice of food in a subject (e.g. a subject with overweight, obesity and/or diabetes) away from unhealthy food, such as having a high glycemic index and/or wherein the mono- and/or di-saccharides constitute a large proportion of the total amount of carbohydrates in said food and/or wherein a large proportion of the total amount of energy stems from fat in said food, each as discussed above.

Further thus, in another aspect, the present invention provides the use of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose)), to regulate or shift food or taste preference or choice of food in a subject (e.g. a subject with overweight, obesity and/or diabetes) towards healthy food, such as having a low glycemic index and/or wherein the mono- and/or di-saccharides constitute a small proportion of the total amount of carbohydrates in said food and/or wherein a small proportion of the total amount of energy stems from fat in said food, each as discussed above.

Further thus, in another aspect, the present invention provides the use of a certain DPP-4 inhibitor (preferably linagliptin), optionally in combination with one or more other active agents (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), to regulate or shift food or taste preference or choice of food in a subject (e.g. a subject with overweight, obesity and/or diabetes) away from unhealthy food, such as having a high glycemic index and/or wherein the mono- and/or di-saccharides constitute a large proportion of the total amount of carbohydrates and/or wherein a large proportion of the total amount of energy stems from fat (each as discussed in more detail above), and towards healthy food, such as having a low glycemic index and/or wherein the mono- and/or di-saccharides constitute a small proportion of the total amount of carbohydrates and/or wherein a small proportion of the total amount of energy stems from fat (each as discussed in more detail above).

For example, in an embodiment, the present invention relates to a method for reducing intake of food by a subject (e.g. a subject with overweight, obesity and/or diabetes), wherein said food has a glycemic index above 60%, or wherein said food has a glycemic index above 40% combined with that more than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method for reducing intake of food by a subject (e.g. a subject with overweight, obesity and/or diabetes), wherein mono- and/or di-saccharides together in said food constitute more than 25% of the total amount of carbohydrate in said food, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method for reducing intake of food by a subject (e.g. a subject with overweight, obesity and/or diabetes), wherein mono- and/or di-saccharides together in said food constitute more than 25% of the total amount of carbohydrate in said food combined with that more than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method of increasing intake of food in a subject, wherein said food has a glycemic index below 60%, or wherein said food has a glycemic index below 40% combined with that less than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method of increasing intake of food in a subject wherein mono- and/or di-saccharides together constitute less than 25% of the total amount of carbohydrate in said food, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method of increasing intake of food in a subject wherein mono- and/or di-saccharides together constitute less than 25% of the total amount of carbohydrate in said food combined with that less than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method of treating a subject with an abnormal or excessive intake of food wherein the glycemic index is above 60%, or wherein the glycemic index is above 40% combined with that more than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method of treating a subject with an abnormal or excessive intake of food wherein the mono- and/or di-saccharides together constitute more than 25% of the total amount of carbohydrates, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to a method of treating a subject with an abnormal or excessive intake of food wherein the mono- and/or di-saccharides together constitute more than 25% of the total amount of carbohydrates combined with that more than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to method of regulating or shifting food or taste preference or choice of food in a subject (e.g. a subject with overweight, obesity and/or diabetes) away from food wherein the glycemic index is above 60%, or wherein the glycemic index is above 40% combined with that more than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to method of regulating or shifting food or taste preference or choice of food in a subject (e.g. a subject with overweight, obesity and/or diabetes) away from food wherein the mono- and/or di-saccharides together constitute more than 25% of the total amount of carbohydrates, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to method of regulating or shifting food or taste preference or choice of food in a subject (e.g. a subject with overweight, obesity and/or diabetes) away from food wherein the mono- and/or di-saccharides together constitute more than 25% of the total amount of carbohydrates combined with that more than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to method of regulating or shifting food or taste preference or choice of food in a subject (e.g. a subject with overweight, obesity and/or diabetes) towards food wherein the glycemic index is below 60%, or wherein the glycemic index is below 40% combined with that less than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to method of regulating or shifting food or taste preference or choice of food in a subject (e.g. a subject with overweight, obesity and/or diabetes) towards food wherein the mono- and/or di-saccharides together constitute less than 25% of the total amount of carbohydrates, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

In another embodiment, the invention relates to method of regulating or shifting food or taste preference or choice of food in a subject (e.g. a subject with overweight, obesity and/or diabetes) towards food wherein the mono- and/or di-saccharides together constitute less than 25% of the total amount of carbohydrates combined with that less than 30% of the total amount of energy stems from fat, said method comprising administering to said subject an effective amount of a certain DPP-4 inhibitor (preferably linagliptin), optionally in combination with another active agent (such as e.g. a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose).

The amount of energy in food is typically quoted in calories or joules, and it can be measured by burning the food, e.g. in a bomb calorimeter. The amount of energy attributable to fat can be determined by multiplying the amount of fat in the food, analysed as discussed above, with 38 kJ/g.

It is well-known that many people prefer sweet and/or fatty food because they think it has a better taste. Accordingly, the present invention also provides a method a regulating taste preferences, and in particular regulating taste preferences away from sweet and fatty food, said method comprising the administration of an effective amount of a certain DPP-4 inhibitor (preferably linagliptin), optionally in combination with one or more other active agents.

It is quite clear that the western world life style is not healthy as evidenced by the increase in obesity with all its pathological consequences, such as diabetes and diabetic complications, and in that sense the life style must be regarded as abnormal. Accordingly, in one embodiment, the present invention relates to a method of normalising lifestyle, and in particular the food preference, said method comprising the administration of an effective amount of a certain DPP-4 inhibitor (preferably linagliptin), optionally in combination with one or more other active agents.

In one embodiment, the subject to be treated has an increased appetite, hunger or craving for sweet or fat food. This may be related to e.g. stress, quit of smoking, pregnancy, premenstrual tension, or it can be ascribed physiological problems or diseases, such as binge eating, compulsive eating habits and Seasonal Affective Disorder.

Binge eating disorder (BED) is a fairly new diagnosable disorder—see e.g. Int. J. Obesity, 2002, 26, 299-307 and Curr. Opin. Psychiatry, 17, 43-48, 2004. BED is characterised by binge eating episodes as is bulimia nervosa (BN). However, subjects with BED do not, contrary to patients with BN, engage in compensatory behaviors, such as e.g. self-induced vomiting, excessive exercise, and misuse of laxatives, diuretics or enemas. Studies have shown that 1-3% of the general population suffers from BED, whereas a higher prevalence (up to 25-30%) has been reported for obese patients [Int. J. Obesity, 2002, 26, 299-307]. These numbers show that BED subjects may or may not be obese, and that obese patients may or may not have BED, i.e. that the cause of the obesity is BED. However, a proportion of subjects with BED eventually becomes obese due to the excess calorie intake. Laboratory studies have shown that BED patients consumed more dessert and snack (rich in fat and poor in proteins) than did an obese control group [Int. J. Obesity, 2002, 26, 299-307], and the method of the present invention is thus believed to be suited for treatment of BED. In one embodiment, the invention relates to a method or treating BED in a subject, the method comprising administering to said subject an effective amount of a certain DPP-4 inhibitor (preferably linagliptin), optionally in combination with one or more other active agents. In particular, said subject is obese.

Bulimia nervosa (BN) is characterised by the same binge eating episodes as is BED, however, BN is, however, also characterised by the above mentioned compensatory behavior. A proportion of subjects with BN will eventually become obese to the extent that the compensatory behavior cannot fully compensate the excess calorie intake. Studies have compared binges of patients with BN and with BED concluding that binges in subjects with BN were higher in carbohydrates and sugar content than those of subjects with BED. No difference was, however, found in the number of consumed calories [Int. J. Obesity, 2002, 26, 299-307]. The methods of the present invention is therefore believed to be suited for the treatment of BN.

In one embodiment, the invention relates to a method of treating BN in a subject, the method comprising administering to said subject an effective amount of a certain DPP-4 inhibitor (preferably linagliptin), optionally in combination with one or more other active agents. In particular, said subject is obese.

Craving for food or the intense desire to eat a particular food is normally associated with energy dense food, such as fatty or carbohydrate-rich food [Appetite, 17, 177-185, 1991; Appetite, 17, 167-175, 1991]. Examples of such foods include chocolate, biscuits, cakes and snacks. A proportion of food cravers eventually become obese due to the excess calorie intake. The methods of the present invention are believed to be suited for the treatment of food craving, in particular craving for fatty or carbohydrate-rich food.

In one embodiment, the invention relates to a method of treating food craving, such as craving for fatty or carbohydrate-rich food, such as chocolate craving in a subject, the method comprising administering to said subject an effective amount of a certain DPP-4 inhibitor (preferably linagliptin), optionally in combination with one or more other active agents. In particular, said subject is obese.

A snack is typically a light, casual, hurried convenience meal eaten between real meals. Snacks are typically fatty and carbohydrate-rich. Studies have shown that there is an increasing prevalence of snacking, especially among US children, and that snacking is a significant cause for the increase in BMI in e.g. children [J. Pediatrics, 138, 493-498, 2001; Obes. Res., 11, 143-151, 2003]. A shift towards more healthy snacks could probably arrest or change the increase in BMI which has taken place over the last years. Data in shown here illustrate that a certain DPP-4 inhibitor, optionally in combination with one or more other active agents, is capable of shifting food preferences from fatty and carbohydrate-rich food to low-fat glycemic index low food. The certain DPP-4 inhibitor, optionally in combination with one or more other active agents, is therefore useful in diminishing the amount of snacking or in changing the preference of snack to more healthy snack.

In one embodiment, the invention relates to a method of changing the snack preference in a subject to low fat, glycemic index low snack, the method comprising administering to said subject an effective amount of a certain DPP-4 inhibitor (preferably linagliptin), optionally in combination with one or more other active agents. In particular, said subject is obese.

In one embodiment, the invention provides a method of lowering the amount a snack intake (“snacking”) of a subject, the method comprising administering to said subject an effective amount of a certain DPP-4 inhibitor (preferably linagliptin), optionally in combination with one or more other active agents. In particular, said subject is obese.

According to the above discussion, the certain DPP-4 inhibitor (preferably linagliptin), optionally in combination with one or more other active agents (such as e.g. including a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose), is believed to be useful in the treatment of obesity or overweight, wherein the obesity or overweight is caused by BED, BN, food craving (e.g. chocolate craving) or snacking, or over-eating.

The subject of the present invention can in principle be any animal, in particular mammals, such as humans, pet animals, such as cats and dogs, and zoo animals, such as elephants, giraffes, lions and snakes; with humans being preferred.

Type 2 diabetes mellitus is a common chronic and progressive disease arising from a complex pathophysiology involving the dual endocrine effects of insulin resistance and impaired insulin secretion with the consequence not meeting the required demands to maintain plasma glucose levels in the normal range. This leads to chronic hyperglycaemia and its associated micro- and macrovascular complications or chronic damages, such as e.g. diabetic nephropathy, retinopathy or neuropathy, or macrovascular (e.g. cardio- or cerebrovascular) complications. The vascular disease component plays a significant role, but is not the only factor in the spectrum of diabetes associated disorders. The high frequency of complications leads to a significant reduction of life expectancy. Diabetes is currently the most frequent cause of adult-onset loss of vision, renal failure, and amputation in the Industrialized World because of diabetes induced complications and is associated with a two to five fold increase in cardiovascular disease risk.

Furthermore, diabetes (particularly type 2 diabetes) is often coexistent and interrelated with obesity and these two conditions together impose a particularly complex therapeutic challenge. Because of the effects of obesity on insulin resistance, weight loss and its maintenance is an important therapeutic objective in overweight or obese individuals with prediabetes, metabolic syndrome or diabetes. Studies have been demonstrated that weight reduction in subjects with type 2 diabetes is associated with decreased insulin resistance, improved measures of glycemia and lipemia, and reduced blood pressure. Maintenance of weight reduction over longer term is considered to improve glycemic control and prevent diabetic complications (e.g. reduction of risk for cardiovascular diseases or events). Thus, weight loss is recommended for all overweight or obese individuals who have or are at risk for diabetes. However, obese patients with type 2 diabetes have much greater difficulty losing weight and maintain the reduced weight than the general non-diabetic population.

In the present context, obese or obesity implies an excess of adipose tissue. In this context obesity is best viewed as any degree of excess adiposity that imparts a health risk. The distinction between normal and obese individuals can only be approximated, but the health risk imparted by obesity is probably a continuum with increasing adiposity. In a broad meaning, individuals with a body mass index (BMI=body weight in kilograms divided by the square of the height in meters) above 25 (or ≧30 kg/m² or ≧27 kg/m²) may be regarded as obese.

Overweight may be defined as the condition wherein the individual has a body mass index (BMI) greater than or 25 kg/m² and less than 30 kg/m². The terms “overweight” and “pre-obese” are used interchangeably.

Obesity may be also defined as the condition wherein the individual has a BMI equal to or greater than 30 kg/m². According to a WHO definition the term obesity may be categorized as follows: class I obesity is the condition wherein the BMI is equal to or greater than 30 kg/m² but lower than 35 kg/m²; class II obesity is the condition wherein the BMI is equal to or greater than 35 kg/m² but lower than 40 kg/m²; class III obesity (extreme obesity) is the condition wherein the BMI is equal to or greater than 40 kg/m². Obesity may include e.g. visceral or abdominal obesity.

Visceral obesity may be defined as the condition wherein a waist-to-hip ratio of greater than or equal to 1.0 in men and 0.8 in women is measured. It defines the risk for insulin resistance and the development of pre-diabetes.

Abdominal obesity may usually be defined as the condition wherein the waist circumference is >40 inches or 102 cm in men, and is >35 inches or 94 cm in women. With regard to a Japanese ethnicity or Japanese patients abdominal obesity may be defined as waist circumference ≧85 cm in men and ≧90 cm in women (see e.g. investigating committee for the diagnosis of metabolic syndrome in Japan).

The treatment of type 2 diabetes typically begins with diet and exercise, followed by oral antidiabetic monotherapy, and although conventional monotherapy may initially control blood glucose in some patients, it is however associated with a high secondary failure rate. The limitations of single-agent therapy for maintaining glycemic control may be overcome, at least in some patients, and for a limited period of time by combining multiple drugs to achieve reductions in blood glucose that cannot be sustained during long-term therapy with single agents. Available data support the conclusion that in most patients with type 2 diabetes current monotherapy will fail and treatment with multiple drugs will be required. But, because type 2 diabetes is a progressive disease, even patients with good initial responses to conventional combination therapy will eventually require an increase of the dosage or further treatment with insulin because the blood glucose level is very difficult to maintain stable for a long period of time. Although existing combination therapy has the potential to enhance glycemic control, it is not without limitations (especially with regard to long term efficacy). Further, traditional therapies may show an increased risk for side effects, such as hypoglycemia or weight gain, which may compromise their efficacy and acceptability.

Thus, for many patients, these existing drug therapies result in progressive deterioration in metabolic control despite treatment and do not sufficiently control metabolic status especially over long-term and thus fail to achieve and to maintain glycemic control in advanced, progressed or late stage type 2 diabetes, including diabetes with inadequate glycemic control despite conventional oral and/or non-oral antidiabetic medication.

Therefore, although intensive treatment of hyperglycemia can reduce the incidence of chronic damages, many patients with diabetes remain inadequately treated, partly because of limitations in long term efficacy, tolerability and dosing inconvenience of conventional antihyperglycemic therapies.

In addition, obesity, overweight or weight gain (e.g. as side or adverse effect of some conventional antidiabetic medications) further complicates the treatment of diabetes and its microvascular or macrovascular complications.

This high incidence of therapeutic failure is a major contributor to the high rate of long-term hyperglycemia-associated complications or chronic damages (including micro- and makrovascular complications such as e.g. diabetic nephrophathy, retinopathy or neuropathy, or cerebro- or cardiovascular complications such as e.g. myocardial infarction, stroke or death) in patients with diabetes.

Oral antidiabetic drugs conventionally used in therapy (such as e.g. first- or second-line, and/or mono- or (initial or add-on) combination therapy) include, without being restricted thereto, metformin, sulphonylureas, thiazolidinediones, glinides and α-glucosidase inhibitors.

Non-oral (typically injected) antidiabetic drugs conventionally used in therapy (such as e.g. first- or second-line, and/or mono- or (initial or add-on) combination therapy) include, without being restricted thereto, GLP-1 or GLP-1 analogues, and insulin or insulin analogues.

However, the use of these conventional antidiabetic or antihyperglycemic agents can be associated with various adverse effects. For example, metformin can be associated with lactic acidosis or gastrointestinal side effects; sulfonylureas, glinides and insulin or insulin analogues can be associated with hypoglycemia and weight gain; thiazolidinediones can be associated with edema, bone fracture, weight gain and heart failure/cardiac effects; and alpha-glucosidase blockers and GLP-1 or GLP-1 analogues can be associated with gastrointestinal adverse effects (e.g. dyspepsia, flatulence or diarrhea, or nausea or vomiting).

Therefore, it remains a need in the art to provide efficacious, safe and tolerable antidiabetic therapies.

Further, within the therapy of type 2 diabetes, it is a need for treating the condition effectively, avoiding the complications inherent to the condition, and delaying disease progression.

Further, within the therapy of type 2 diabetes, it is a need for sustained improvements in diabetic phenotype, glycemic and/or metabolic control, and/or (blood) glucose profile (preferably over long-term and/or during chronic treatment).

Furthermore, it remains a need that antidiabetic treatments not only prevent the long-term complications often found in advanced stages of diabetes disease, but also are a therapeutic option in those diabetes patients who have developed or are at risk of developing complications, such as renal impairment.

Moreover, it remains a need to provide prevention or reduction of risk for adverse effects associated with conventional antidiabetic therapies.

Further, it remains a need in the art to provide efficacious, safe and tolerable therapies for obesity patients with or without diabetes, particularly for reducing, facilitating a reduction, controlling, maintaining and/or preventing an increase of body weight in such patients.

Further, within the management of the dual epidemic of diabetes and obesity (“diabesity”), it is an objective to find therapies which are safe, tolerable and effective in the treatment or prevention of these conditions together, particularly in achieving long term weight reduction and weight control, and improving glycemic control.

Within the scope of the present invention it has now been found that a certain DPP-4 inhibitor (preferably linagliptin) as defined herein as well as pharmaceutical combinations, compositions, uses or methods according to this invention of that DPP-4 inhibitor (preferably linagliptin) optionally in combination with one or more other active agents (such as e.g. including a GLP-1 receptor agonist, particularly a short acting GLP-1, GLP-1 mimetic or GLP-1 analogue, such as exenatide or native GLP-1, and/or such as e.g. including an alpha-glucosidase inhibitor, such as voglibose) as defined herein have properties, which make them suitable for the purpose of this invention and/or for fulfilling one or more of the needs mentioned herein.

The enzyme DPP-4 (dipeptidyl peptidase IV) also known as CD26 is a serine protease known to lead to the cleavage of a dipeptide from the N-terminal end of a number of proteins having at their N-terminal end a prolin or alanin residue. Due to this property DPP-4 inhibitors interfere with the plasma level of bioactive peptides including the peptide GLP-1 and are considered to be promising drugs for the treatment of diabetes mellitus.

For example, DPP-4 inhibitors and their uses are disclosed in WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769, WO2007/014886; WO 2004/050658, WO 2004/111051, WO 2005/058901, WO 2005/097798; WO 2006/068163, WO 2007/071738, WO 2008/017670; WO 2007/128721, WO 2007/128724, WO 2007/128761, or WO 2009/121945.

A DPP-4 inhibitor within the meaning of the present invention includes, without being limited to, any of those DPP-4 inhibitors mentioned hereinabove and herein below, preferably orally and/or subcutaneously active DPP-4 inhibitors.

A GLP-1 receptor agonist within the meaning of this invention includes, without being limited, exogenous GLP-1 (natural or synthetic), GLP-1 analogue, GLP-1 mimetic and any other substance (whether peptidic or non-peptidic, e.g. small molecule) which promotes signalling through the GLP-1 receptor; preferably included is any short acting representative thereof, such as e.g. a short acting GLP-1, GLP-1 mimetic or GLP-1 analogue, such as e.g. exenatide or native GLP-1.

Within the context of this invention, a short acting GLP-1, GLP-1 mimetic or GLP-1 analogue refers to those of such agents having a short half life (or to be administered subcutaneously at least twice daily), such as e.g. exendin-4 or exenatide, or native GLP-1.

Accordingly, a short acting GLP-1 receptor agonist may be herein referred to as such agent having duration of action of <24 h, or having a short half life of about below 13 h, below 10 h, below 5 h, or below 2.5 h (e.g. about 2.4 h or even below), or to be administered subcutaneously at least twice daily, such as e.g. exenatide or native GLP-1.

All of these agents, as far as they exhibit the desired property and function, are contemplated and included within the scope of this invention.

In a first embodiment (embodiment A), a DPP-4 inhibitor in the context of the present invention is any DPP-4 inhibitor of formula (I)

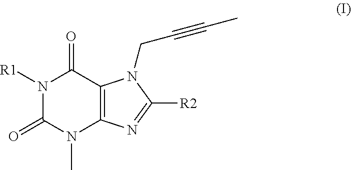

or formula (II)

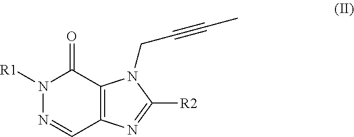

or formula (III)

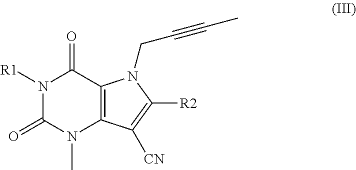

or formula (IV)

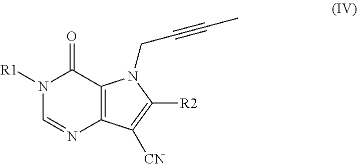

wherein R1 denotes ([1,5]naphthyridin-2-yl)methyl, (quinazolin-2-yl)methyl, (quinoxalin-6-yl)methyl, (4-methyl-quinazolin-2-yl)methyl, 2-cyano-benzyl, (3-cyano-quinolin-2-yl)methyl, (3-cyano-pyridin-2-yl)methyl, (4-methyl-pyrimidin-2-yl)methyl, or (4,6-dimethyl-pyrimidin-2-yl)methyl and R2 denotes 3-(R)-amino-piperidin-1-yl, (2-amino-2-methyl-propyl)-methylamino or (2-(S)-amino-propyl)-methylamino,
or its pharmaceutically acceptable salt.

Regarding the first embodiment (embodiment A), preferred DPP-4 inhibitors are any or all of the following compounds and their pharmaceutically acceptable salts:
1-[(4-methyl-quinazolin-2-yl)methyl]-3-methyl-7-(2-butyn-1-yl)-8-(3-(R)-amino-piperidin-1-yl)-xanthine (compare WO 2004/018468, example 2(142)):

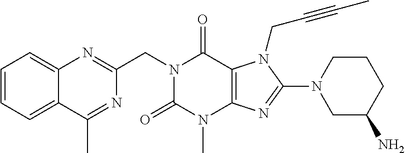 [SMILES: CC#CCn1c(N2CCC[C@@H](N)C2)nc2c1c(=O)n(Cc1nc(C)c3ccccc3n1)c(=O)n2C]

1-[([1,5]naphthyridin-2-yl)methyl]-3-methyl-7-(2-butyn-1-yl)-8-((R)-3-amino-piperidin-1-yl)-xanthine (compare WO 2004/018468, example 2(252)):

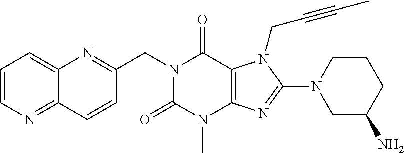 [SMILES: CC#CCn1c(N2CCC[C@@H](N)C2)nc2c1c(=O)n(Cc1ccc3ncccc3n1)c(=O)n2C]

1-[(Quinazolin-2-yl)methyl]-3-methyl-7-(2-butyn-1-yl)-8-((R)-3-amino-piperidin-1-yl)-xanthine (compare WO 2004/018468, example 2(80)):

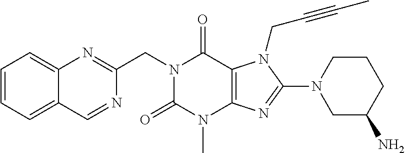 [SMILES: CC#CCn1c(N2CCC[C@@H](N)C2)nc2c1c(=O)n(Cc1ncc3ccccc3n1)c(=O)n2C]

2-((R)-3-Amino-piperidin-1-yl)-3-(but-2-yinyl)-5-(4-methyl-quinazolin-2-ylmethyl)-3,5-dihydro-imidazo[4,5-d]pyridazin-4-one (compare WO 2004/050658, example 136):

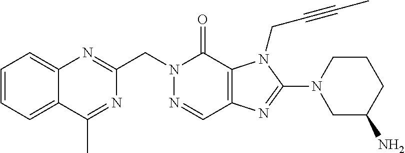 [SMILES: CC#CCn1c(N2CCC[C@@H](N)C2)nc2cnn(Cc3nc(C)c4ccccc4n3)c(=O)c21]

1-[(4-Methyl-quinazolin-2-yl)methyl]-3-methyl-7-(2-butyin-1-yl)-8-[(2-amino-2-methyl-propyl)-methylamino]-xanthine (compare WO 2006/029769, example 2(1)):

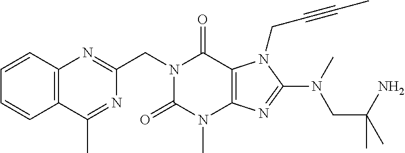 [SMILES: CC#CCn1c(N(C)CC(C)(C)N)nc2c1c(=O)n(Cc1nc(C)c3ccccc3n1)c(=O)n2C]

1-[(3-Cyano-quinolin-2-yl)methyl]-3-methyl-7-(2-butyn-1-yl)-8-((R)-3-amino-piperidin-1-yl)-xanthine (compare WO 2005/085246, example 1(30)):

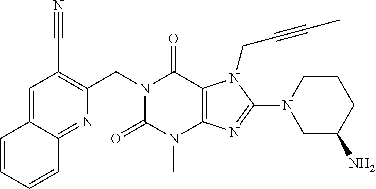 [SMILES: CC#CCn1c(N2CCC[C@@H](N)C2)nc2c1c(=O)n(Cc1nc3ccccc3cc1C#N)c(=O)n2C]

1-(2-Cyano-benzyl)-3-methyl-7-(2-butyn-1-yl)-8-((R)-3-amino-piperidin-1-yl)-xanthine (compare WO 2005/085246, example 1(39)):

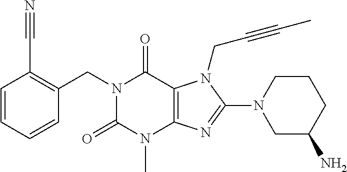 [SMILES: CC#CCn1c(N2CCC[C@@H](N)C2)nc2c1c(=O)n(Cc1ccccc1C#N)c(=O)n2C]

1-[(4-Methyl-quinazolin-2-yl)methyl]-3-methyl-7-(2-butyn-1-yl)-8-[(S)-(2-amino-propyl)-methylamino]-xanthine (compare WO 2006/029769, example 2(4)):

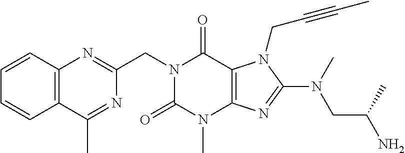 [SMILES: CC#CCn1c(N(C)C[C@H](C)N)nc2c1c(=O)n(Cc1nc(C)c3ccccc3n1)c(=O)n2C]

1-[(3-Cyano-pyridin-2-yl)methyl]-3-methyl-7-(2-butyn-1-yl)-8-((R)-3-amino-piperidin-1-yl)-xanthine (compare WO 2005/085246, example 1(52)):

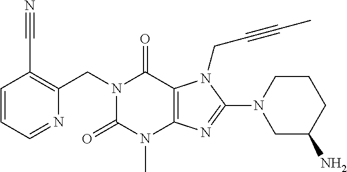 [SMILES: CC#CCn1c(N2CCC[C@@H](N)C2)nc2c1c(=O)n(Cc1ncccc1C#N)c(=O)n2C]

1-[(4-Methyl-pyrimidin-2-yl)methyl]-3-methyl-7-(2-butyn-1-yl)-8-((R)-3-amino-piperidin-1-yl)-xanthine (compare WO 2005/085246, example 1(81)):

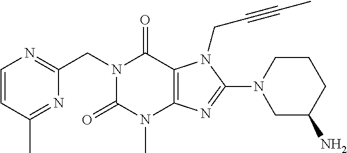 [SMILES: CC#CCn1c(N2CCC[C@@H](N)C2)nc2c1c(=O)n(Cc1nccc(C)n1)c(=O)n2C]

1-[(4,6-Dimethyl-pyrimidin-2-yl)methyl]-3-methyl-7-(2-butyn-1-yl)-8-((R)-3-amino-piperidin-1-yl)-xanthine (compare WO 2005/085246, example 1(82)):

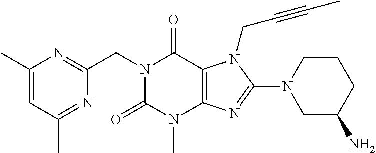 [SMILES: CC#CCn1c(N2CCC[C@@H](N)C2)nc2c1c(=O)n(Cc1nc(C)cc(C)n1)c(=O)n2C]

1-[(Quinoxalin-6-yl)methyl]-3-methyl-7-(2-butyn-1-yl)-8-((R)-3-amino-piperidin-1-yl)-xanthine (compare WO 2005/085246, example 1(83)):

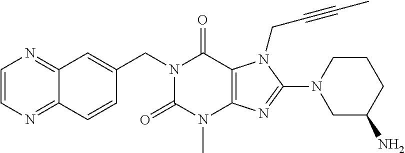 [SMILES: CC#CCn1c(N2CCC[C@@H](N)C2)nc2c1c(=O)n(Cc1ccc3nccnc3c1)c(=O)n2C]

These DPP-4 inhibitors are distinguished from structurally comparable DPP-4 inhibitors, as they combine exceptional potency and a long-lasting effect with favourable pharmacological properties, receptor selectivity and a favourable side-effect profile or bring about unexpected therapeutic advantages or improvements when combined with other pharmaceutical active substances. Their preparation is disclosed in the publications mentioned.

In a second embodiment (embodiment B), a DPP-4 inhibitor in the context of the present invention is a DPP-4 inhibitor selected from the group consisting of sitagliptin, vildagliptin, saxagliptin, alogliptin, gemigliptin, omarigliptin, evogliptin,
(2S)-1-{[2-(5-Methyl-2-phenyl-oxazol-4-yl)-ethylamino]-acetyl}-pyrrolidine-2-carbonitrile,
(2S)-1-{[1,1,-Dimethyl-3-(4-pyridin-3-yl-imidazol-1-yl)-propylamino]-acetyl}-pyrrolidine-2-carbonitrile,
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one,
(3,3-Difluoropyrrolidin-1-yl)-((2S,4S)-4-(4-(pyrimidin-2-yl)piperazin-1-yl)pyrrolidin-2-yl)methanone,
(1((3S,4S)-4-amino-1-(4-(3,3-difluoropyrrolidin-1-yl)-1,3,5-triazin-2-yl)pyrrolidin-3-yl)-5,5-difluoropiperidin-2-one,
(2S,4S)-1-{2-[(3S,1R)-3-(1H-1,2,4-Triazol-1-ylmethyl)cyclopentylamino]-acetyl}-4-fluoropyrrolidine-2-carbonitrile,
(R)-2-[6-(3-Amino-piperidin-1-yl)-3-methyl-2,4-dioxo-3,4-dihydro-2H-pyrimidin-1-ylmethyl]-4-fluoro-benzonitrile,
5-{(S)-2-[2-((S)-2-Cyano-pyrrolidin-1-yl)-2-oxo-ethylamino]-propyl}-5-(1H-tetrazol-5-yl)-10,11-dihydro-5H-dibenzo[a,d]cycloheptene-2,8-dicarboxylic acid bis-dimethylamide,
3-{(2S,4S)-4-[4-(3-Methyl-1-phenyl-1H-pyrazol-5-yl)piperazin-1-yl]pyrrolidin-2-ylcarbonyl}thiazolidine,
[(2R)-1-{[(3R)-pyrrolidin-3-ylamino]acetyl}pyrrolidin-2-yl]boronic acid,
(2S,4S)-1-[2-[(4-ethoxycarbonylbicyclo[2.2.2]oct-1-yl)amino]acetyl]-4-fluoropyrrolidine-2-carbonitrile,
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile,
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione, and
(S)-2-methylpyrazolo[1,5-a]primidine-6-carboxylic acid {2-[(2-cyanopyrrolidin-1-yl)-2-oxoethylamino]-2-methylpropyl}amide,
   or its pharmaceutically acceptable salt.

A more preferred DPP-4 inhibitor among the abovementioned DPP-4 inhibitors of embodiment A of this invention is 1-[(4-methyl-quinazolin-2-yl)methyl]-3-methyl-7-(2-butyn-1-yl)-8-(3-(R)-amino-piperidin-1-yl)-xanthine, particularly the free base thereof (which is also known as linagliptin or BI 1356).

As further DPP-4 inhibitors the following compounds can be mentioned:
Sitagliptin (MK-0431) having the structural formula A below is (3R)-3-amino-1-[3-(trifluoromethyl)-5,6,7,8-tetrahydro-5H-[1,2,4]triazolo[4,3-a]pyrazin-7-yl]-4-(2,4,5-trifluorophenyl)butan-1-one, also named (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine,

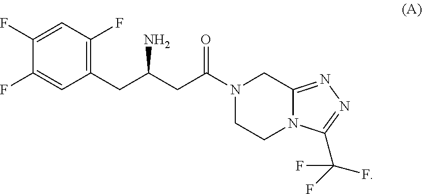 [SMILES: N[C@@H](CC(=O)N1CCn2c(nnc2C(F)(F)F)C1)Cc1cc(F)c(F)cc1F]

In one embodiment, sitagliptin is in the form of its dihydrogenphosphate salt, i.e. sitagliptin phosphate. In a further embodiment, sitagliptin phosphate is in the form of a crystalline anhydrate or monohydrate. A class of this embodiment refers to sitagliptin phosphate monohydrate. Sitagliptin free base and pharmaceutically acceptable salts thereof are disclosed in U.S. Pat. No. 6,699,871 and in Example 7 of WO 03/004498. Crystalline sitagliptin phosphate monohydrate is disclosed in WO 2005/003135 and in WO 2007/050485.

For details, e.g. on a process to manufacture, to formulate or to use this compound or a salt thereof, reference is thus made to these documents.

A tablet formulation for sitagliptin is commercially available under the trade name Januvia®. A tablet formulation for sitagliptin/metformin combination is commercially available under the trade name Janumet®.
Vildagliptin (LAF-237) having the structural formula B below is (2S)-{[(3-hydroxyadamantan-1-yl)amino]acetyl}pyrrolidine-2-carbonitrile, also named (S)-1-[(3-hydroxy-1-adamantyl)amino]acetyl-2-cyano-pyrrolidine,

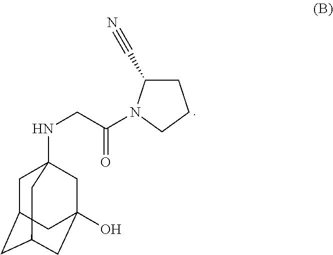 [SMILES: N#C[C@@H]1CCCN1C(=O)CNC12CC3CC(CC(O)(C3)C1)C2]

Vildagliptin is specifically disclosed in U.S. Pat. No. 6,166,063 and in Example 1 of WO 00/34241. Specific salts of vildagliptin are disclosed in WO 2007/019255. A crystalline form of vildagliptin as well as a vildagliptin tablet formulation are disclosed in WO 2006/078593. Vildagliptin can be formulated as described in WO 00/34241 or in WO 2005/067976. A modified release vildagliptin formulation is described in WO 2006/135723.

For details, e.g. on a process to manufacture, to formulate or to use this compound or a salt thereof, reference is thus made to these documents.

A tablet formulation for vildagliptin is expected to be commercially available under the trade name Galvus®. A tablet formulation for vildagliptin/metformin combination is commercially available under the trade name Eucreas®.
Saxagliptin (BMS-477118) having the structural formula C below is (1 S,3S,5S)-2-{(2S)-2-amino-2-(3-hydroxyadamantan-1-yl)acetyl}-2-azabicyclo[3.1.0]hexane-3-carbonitrile, also named (S)-3-hydroxyadamantylglycine-L-cis-4,5-methanoprolinenitrile,

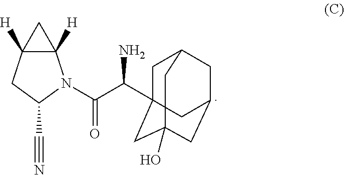 [SMILES: N#C[C@@H]1C[C@@H]2C[C@@H]2N1C(=O)[C@@H](N)C12CC3CC(CC(O)(C3)C1)C2]

Saxagliptin is specifically disclosed in U.S. Pat. No. 6,395,767 and in Example 60 of WO 01/68603.

In one embodiment, saxagliptin is in the form of its HCl salt or its mono-benzoate salt as disclosed in WO 2004/052850. In a further embodiment, saxagliptin is in the form of the free base. In a yet further embodiment, saxagliptin is in the form of the monohydrate of the free base as disclosed in WO 2004/052850. Crystalline forms of the HCl salt and of the free base of saxagliptin are disclosed in WO 2008/131149. A process for preparing saxagliptin is also disclosed in WO 2005/106011 and WO 2005/115982. Saxagliptin can be formulated in a tablet as described in WO 2005/117841.

For details, e.g. on a process to manufacture, to formulate or to use this compound or a salt thereof, reference is thus made to these documents.
Alogliptin (SYR-322) having the structural formula E below is 2-({6-[(3R)-3-aminopiperidin-1-yl]-3-methyl-2,4-dioxo-3,4-dihydro-2H-pyrimidin-1-yl}methyl)benzonitrile

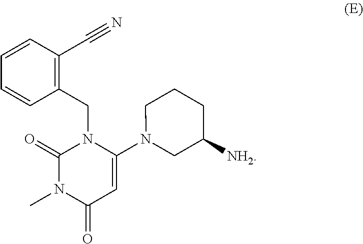 [SMILES: Cn1c(=O)cc(N2CCC[C@@H](N)C2)n(Cc2ccccc2C#N)c1=O]

Alogliptin is specifically disclosed in US 2005/261271, EP 1586571 and in WO 2005/095381. In one embodiment, alogliptin is in the form of its benzoate salt, its hydrochloride salt or its tosylate salt each as disclosed in WO 2007/035629. A class of this embodiment refers to alogliptin benzoate. Polymorphs of alogliptin benzoate are disclosed in WO 2007/035372. A process for preparing alogliptin is disclosed in WO 2007/112368 and, specifically, in WO 2007/035629. Alogliptin (namely its benzoate salt) can be formulated in a tablet and administered as described in WO 2007/033266. A solid preparation of alogliptin/pioglitazone and its preparation and use is described in WO 2008/093882. A solid preparation of alogliptin/metformin and its preparation and use is described in WO 2009/011451.

For details, e.g. on a process to manufacture, to formulate or to use this compound or a salt thereof, reference is thus made to these documents.
(2 S)-1-{[2-(5-Methyl-2-phenyl-oxazol-4-yl)-ethylamino]-acetyl}-pyrrolidine-2-carbonitrile or a pharmaceutically acceptable salt thereof, preferably the mesylate, or (2S)-1-{[1,1,-Dimethyl-3-(4-pyridin-3-yl-imidazol-1-yl)-propylamino]-acetyl}-pyrrolidine-2-carbonitrile or a pharmaceutically acceptable salt thereof:

These compounds and methods for their preparation are disclosed in WO 03/037327. The mesylate salt of the former compound as well as crystalline polymorphs thereof are disclosed in WO 2006/100181. The fumarate salt of the latter compound as well as crystalline polymorphs thereof are disclosed in WO 2007/071576. These compounds can be formulated in a pharmaceutical composition as described in WO 2007/017423.

For details, e.g. on a process to manufacture, to formulate or to use these compounds or salts thereof, reference is thus made to these documents.
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one (also named carmegliptin) or a pharmaceutically acceptable salt thereof:

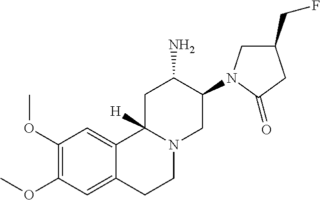 [SMILES: COc1cc2c(cc1OC)[C@@H]1C[C@H](N)[C@@H](N3C[C@@H](CF)CC3=O)CN1CC2]

This compound and methods for its preparation are disclosed in WO 2005/000848. A process for preparing this compound (specifically its dihydrochloride salt) is also disclosed in WO 2008/031749, WO 2008/031750 and WO 2008/055814. This compound can be formulated in a pharmaceutical composition as described in WO 2007/017423.

For details, e.g. on a process to manufacture, to formulate or to use this compound or a salt thereof, reference is thus made to these documents.
(3,3-Difluoropyrrolidin-1-yl)-((2S,4S)-4-(4-(pyrimidin-2-yl)piperazin-1-yl)pyrrolidin-2-yl)methanone (also named gosogliptin) or a pharmaceutically acceptable salt thereof:

This compound and methods for its preparation are disclosed in WO 2005/116014 and U.S. Pat. No. 7,291,618.

For details, e.g. on a process to manufacture, to formulate or to use this compound or a salt thereof, reference is thus made to these documents.
(1((3S,4S)-4-amino-1-(4-(3,3-difluoropyrrolidin-1-yl)-1,3,5-triazin-2-yl)pyrrolidin-3-yl)-5,5-difluoropiperidin-2-one or a pharmaceutically acceptable salt thereof:

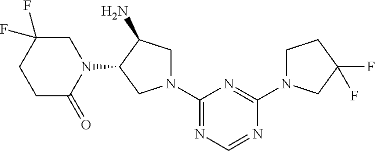 [SMILES: N[C@H]1CN(c2ncnc(N3CCC(F)(F)C3)n2)C[C@@H]1N1CC(F)(F)CCC1=O]

This compound and methods for its preparation are disclosed in WO 2007/148185 and US 20070299076. For details, e.g. on a process to manufacture, to formulate or to use this compound or a salt thereof, reference is thus made to these documents.
(2S,4S)-1-{2-[(3S,1R)-3-(1H-1,2,4-Triazol-1-ylmethyl)cyclopentylamino]-acetyl}-4-fluoropyrrolidine-2-carbonitrile (also named melogliptin) or a pharmaceutically acceptable salt thereof:

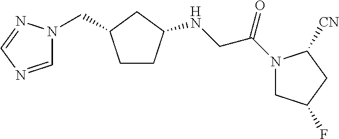 [SMILES: N#C[C@@H]1C[C@H](F)CN1C(=O)CN[C@@H]1CC[C@H](Cn2cncn2)C1]

This compound and methods for its preparation are disclosed in WO 2006/040625 and WO 2008/001195. Specifically claimed salts include the methanesulfonate and p-toluenesulfonate. For details, e.g. on a process to manufacture, to formulate or to use this compound or a salt thereof, reference is thus made to these documents.
(R)-2-[6-(3-Amino-piperidin-1-yl)-3-methyl-2,4-dioxo-3,4-dihydro-2H-pyrimidin-1-ylmethyl]-4-fluoro-benzonitrile or a pharmaceutically acceptable salt thereof:

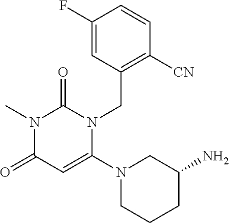 [SMILES: Cn1c(=O)cc(N2CCC[C@@H](N)C2)n(Cc2cc(F)ccc2C#N)c1=O]

This compound and methods for its preparation and use are disclosed in WO 2005/095381, US 2007060530, WO 2007/033350, WO 2007/035629, WO 2007/074884, WO 2007/112368, WO 2008/033851, WO 2008/114800 and WO 2008/114807. Specifically claimed salts include the succinate (WO 2008/067465), benzoate, benzenesulfonate, p-toluenesulfonate, (R)-mandelate and hydrochloride. For details, e.g. on a process to manufacture, to formulate or to use this compound or a salt thereof, reference is thus made to these documents.
5-{(S)-2-[2-((S)-2-Cyano-pyrrolidin-1-yl)-2-oxo-ethylamino]-propyl}-5-(1H-tetrazol-5-yl)-10,11-dihydro-5H-dibenzo[a,d]cycloheptene-2,8-dicarboxylic acid bis-dimethylamide or a pharmaceutically acceptable salt thereof:

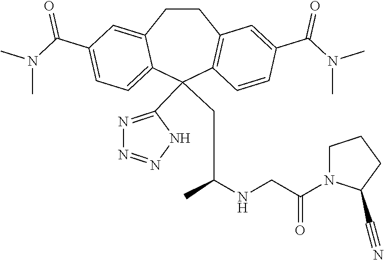 [SMILES: C[C@@H](CC1(c2nnn[nH]2)c2ccc(C(=O)N(C)C)cc2CCc2cc(C(=O)N(C)C)ccc21)NCC(=O)N1CCC[C@H]1C#N]

This compound and methods for its preparation are disclosed in WO 2006/116157 and US 2006/270701. For details, e.g. on a process to manufacture, to formulate or to use this compound or a salt thereof, reference is thus made to these documents.
3-{(2S,4S)-4-[4-(3-Methyl-1-phenyl-1H-pyrazol-5-yl)piperazin-1-yl]pyrrolidin-2-ylcarbonyl}thiazolidine (also named teneligliptin) or a pharmaceutically acceptable salt thereof:

This compound and methods for its preparation are disclosed in WO 02/14271. Specific salts are disclosed in WO 2006/088129 and WO 2006/118127 (including hydrochloride, hydrobromide, inter alia). Combination therapy using this compound is described in WO 2006/129785. For details, e.g. on a process to manufacture, to formulate or to use this compound or a salt thereof, reference is thus made to these documents.
[(2R)-1-{[(3R)-pyrrolidin-3-ylamino]acetyl}pyrrolidin-2-yl]boronic acid (also named dutogliptin) or a pharmaceutically acceptable salt thereof:

This compound and methods for its preparation are disclosed in WO 2005/047297, WO 2008/109681 and WO 2009/009751. Specific salts are disclosed in WO 2008/027273 (including citrate, tartrate). A formulation of this compound is described in WO 2008/144730. A formulation of dutogliptin (as its tartrate salt) with metformin is described in WO 2009/091663. For details, e.g. on a process to manufacture, to formulate or to use this compound or a salt thereof, reference is thus made to these documents.
(2S,4S)-1-[2-[(4-ethoxycarbonylbicyclo[2.2.2]oct-1-yl)amino]acetyl]-4-fluoropyrrolidine-2-carbonitrile (also named bisegliptin) or a pharmaceutically acceptable salt thereof:

This compound and methods for its preparation are disclosed in WO 2005/075421, US 2008/146818 and WO 2008/114857. For details, e.g. on a process to manufacture, to formulate or to use this compound or a salt thereof, reference is thus made to these documents.
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile or a pharmaceutically acceptable salt thereof, or 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione or a pharmaceutically acceptable salt thereof:

These compounds and methods for their preparation are disclosed in WO 2009/084497 and WO 2006/068163, respectively. Combination therapy using the latter of these two compounds is described in WO 2009/128360. For details, e.g. on a process to manufacture, to formulate or to use these compounds or salts thereof, reference is thus made to these documents.
(S)-2-methylpyrazolo[1,5-a]primidine-6-carboxylic acid {2-[(2-cyanopyrrolidin-1-yl)-2-oxoethylamino]-2-methylpropyl}amide (also named anagliptin) or a pharmaceutically acceptable salt:

This compound and methods for its preparation are disclosed in WO 2004/067509. Combination therapy using this compound is described in WO 2009/139362. For details, e.g. on a process to manufacture, to formulate or to use this compound or a salt thereof, reference is thus made to these documents.

Preferably the DPP-4 inhibitor of this invention is selected from the group consisting of linagliptin, sitagliptin, vildagliptin, alogliptin, saxagliptin, teneligliptin, anagliptin, gemigliptin and dutogliptin, or a pharmaceutically acceptable salt of one of the herein mentioned DPP-4 inhibitors, or a prodrug thereof.

A particularly preferred DPP-4 inhibitor to be emphasized within the present invention is linagliptin. The term “linagliptin” as employed herein refers to linagliptin or a pharmaceutically acceptable salt thereof, including hydrates and solvates thereof, and crystalline forms thereof, preferably linagliptin refers to 1-[(4-methyl-quinazolin-2-yl)methyl]-3-methyl-7-(2-butyn-1-yl)-8-(3-(R)-amino-piperidin-1-yl)-xanthine. Crystalline forms are described in WO 2007/128721. Methods for the manufacture of linagliptin are described in the patent applications WO 2004/018468 and WO 2006/048427 for example. Linagliptin is distinguished from structurally comparable DPP-4 inhibitors, as it combines exceptional potency and a long-lasting effect with favourable pharmacological properties, receptor selectivity and a favourable side-effect profile or bring about unexpected therapeutic advantages or improvements in mono- or dual or triple combination therapy.

For avoidance of any doubt, the disclosure of each of the foregoing and following documents cited above in connection with the specified DPP-4 inhibitors is specifically incorporated herein by reference in its entirety.

An embodiment of this invention refers to a DPP-4 inhibitor suitable for use in the treatment and/or prevention of metabolic diseases (particularly type 2 diabetes mellitus) in patients, wherein said patients further suffering from renal disease, renal dysfunction or renal impairment, particularly characterized in that said DPP-4 inhibitor is administered to said patients in the same dose levels as to patients with normal renal function, thus e.g. said DPP-4 inhibitor does not require downward dosing adjustment for impaired renal function.

For example, a DPP-4 inhibitor according to this invention (especially one which may be suited for patients with impaired renal function) may be such an oral DPP-4 inhibitor, which and whose active metabolites have preferably a relatively wide (e.g. about >100 fold) therapeutic window and/or, especially, that are primarily eliminated via hepatic metabolism or biliary excretion (preferably without adding additional burden to the kidney).

In more detailed example, a DPP-4 inhibitor according to this invention (especially one which may be suited for patients with impaired renal function) may be such an orally administered DPP-4 inhibitor, which has a relatively wide (e.g. >100 fold) therapeutic window (preferably a safety profile comparable to placebo) and/or which fulfils one or more of the following pharmacokinetic properties (preferably at its therapeutic oral dose levels):
The DPP-4 inhibitor is substantially or mainly excreted via the liver (e.g. >80% or even >90% of the administered oral dose), and/or for which renal excretion represents no substantial or only a minor elimination pathway (e.g. <10%, preferably <7%, of the administered oral dose measured, for example, by following elimination of a radiolabelled carbon (¹⁴C) substance oral dose);
The DPP-4 inhibitor is excreted mainly unchanged as parent drug (e.g. with a mean of >70%, or >80%, or, preferably, 90% of excreted radioactivity in urine and faeces after oral dosing of radiolabelled carbon (¹⁴C) substance), and/or which is eliminated to a non-substantial or only to a minor extent via metabolism (e.g. <30%, or <20%, or, preferably, 10%);
The (main) metabolite(s) of the DPP-4 inhibitor is/are pharmacologically inactive. Such as e.g. the main metabolite does not bind to the target enzyme DPP-4 and, optionally, it is rapidly eliminated compared to the parent compound (e.g. with a terminal half-life of the metabolite of ≦20 h, or, preferably, ≦about 16 h, such as e.g. 15.9 h).

In one embodiment, the (main) metabolite in plasma (which may be pharmacologically inactive) of a DPP-4 inhibitor having a 3-amino-piperidin-1-yl substituent is such a derivative where the amino group of the 3-amino-piperidin-1-yl moiety is replaced by a hydroxyl group to form the 3-hydroxy-piperidin-1-yl moiety (e.g. the 3-(S)-hydroxy-piperidin-1-yl moiety, which is formed by inversion of the configuration of the chiral center).

Further properties of a DPP-4 inhibitor according to this invention may be one or more of the following: Rapid attainment of steady state (e.g. reaching steady state plasma levels (>90% of the steady state plasma concentration) between second and fifth day of treatment with therapeutic oral dose levels), little accumulation (e.g. with a mean accumulation ratio R_{A,AUC}≦1.4 with therapeutic oral dose levels), and/or preserving a long-lasting effect on DPP-4 inhibition, preferably when used once-daily (e.g. with almost complete (>90%) DPP-4 inhibition at therapeutic oral dose levels, >80% inhibition over a 24 h interval after once-daily intake of therapeutic oral drug dose), significant decrease in 2 h postprandial blood glucose excursions by ≧80% (already on first day of therapy) at therapeutic dose levels, and cumulative amount of unchanged parent compound excreted in urine on first day being below 1% of the administered dose and increasing to not more than about 3-6% in steady state.

Thus, for example, a DPP-4 inhibitor according to this invention may be characterized in that said DPP-4 inhibitor has a primarily non-renal route of excretion, i.e. said DPP-4 inhibitor is excreted to a non-substantial or only to a minor extent (e.g. <10%, preferably <7%, e.g. about 5%, of administered oral dose, preferably of oral therapeutic dose) via the kidney (measured, for example, by following elimination of a radiolabelled carbon (¹⁴C) substance oral dose).

Further, a DPP-4 inhibitor according to this invention may be characterized in that said DPP-4 inhibitor is excreted substantially or mainly via the liver, bile or faeces (measured, for example, by following elimination of a radiolabelled carbon (¹⁴C) substance oral dose).

Further, a DPP-4 inhibitor according to this invention may be characterized in that said DPP-4 inhibitor is excreted mainly unchanged as parent drug (e.g. with a mean of >70%, or >80%, or, preferably, 90% of excreted radioactivity in urine and faeces after oral dosing of radiolabelled carbon (¹⁴C) substance),

said DPP-4 inhibitor is eliminated to a non-substantial or only to a minor extent via metabolism, and/or
the main metabolite of said DPP-4 inhibitor is pharmacologically inactive or has a relatively wide therapeutic window.

Further, a DPP-4 inhibitor according to this invention may be characterized in that said DPP-4 inhibitor does not significantly impair glomerular and/or tubular function of a type 2 diabetes patient with chronic renal insufficiency (e.g. mild, moderate or severe renal impairment or end stage renal disease), and/or

said DPP-4 inhibitor trough levels in the blood plasma of type 2 diabetes patients with mild or moderate renal impairment are comparable to the levels in patients with normal renal function, and/or
said DPP-4 inhibitor does not require to be dose-adjusted in a type 2 diabetes patient with impaired renal function (e.g. mild, moderate or severe renal impairment or end stage renal disease, preferably regardless of the stage of renal impairment).

Further, a DPP-4 inhibitor according to this invention may be characterized in that said DPP-4 inhibitor provides its minimally effective dose at that dose that results in >50% inhibition of DPP-4 activity at trough (24 h after last dose) in >80% of patients, and/or said DPP-4 inhibitor provides its fully therapeutic dose at that dose that results in >80% inhibition of DPP-4 activity at trough (24 h after last dose) in >80% of patients.

Further, a DPP-4 inhibitor according to this invention may be characterized in that being suitable for use in type 2 diabetes patients who are with diagnosed renal impairment or complication and/or who are at risk of developing renal complications, e.g. patients with or at risk of diabetic nephropathy (including chronic and progressive renal insufficiency, albuminuria, proteinuria, fluid retention in the body (edema) and/or hypertension).

In the monitoring of the treatment of diabetes mellitus the HbA1c value, the product of a non-enzymatic glycation of the haemoglobin B chain, is of exceptional importance. As its formation depends essentially on the blood sugar level and the life time of the erythrocytes the HbA1c in the sense of a “blood sugar memory” reflects the average blood sugar level of the preceding 4-12 weeks. Diabetic patients whose HbA1c level has been well controlled over a long time by more intensive diabetes treatment (i.e. <6.5% of the total haemoglobin in the sample) are significantly better protected from diabetic microangiopathy. The available treatments for diabetes can give the diabetic an average improvement in their HbA1c level of the order of 1.0-1.5%. This reduction in the HbA1C level is not sufficient in all diabetics to bring them into the desired target range of <7.0%, preferably <6.5% and more preferably <6% HbA1c.

Within the meaning of this invention, inadequate or insufficient glycemic control means in particular a condition wherein patients show HbA1c values above 6.5%, in particular above 7.0%, even more preferably above 7.5%, especially above 8%. An embodiment of patients with inadequate or insufficient glycemic control include, without being limited to, patients having a HbA1c value from 7.5 to 10% (or, in another embodiment, from 7.5 to 11%). A special sub-embodiment of inadequately controlled patients refers to patients with poor glycemic control including, without being limited, patients having a HbA1c value 9%.

Within glycemic control, in addition to improvement of the HbA1c level, other recommended therapeutic goals for type 2 diabetes mellitus patients are improvement of fasting plasma glucose (FPG) and of postprandial plasma glucose (PPG) levels to normal or as near normal as possible. Recommended desired target ranges of preprandial (fasting) plasma glucose are 70-130 mg/dL (or 90-130 mg/dL) or <110 mg/dL, and of two-hour postprandial plasma glucose are <180 mg/dL or <140 mg/dL.

In one embodiment, diabetes patients within the meaning of this invention may include patients who have not previously been treated with an antidiabetic drug (drug-naïve patients). Thus, in an embodiment, the therapies described herein may be used in naïve patients. In another embodiment, diabetes patients within the meaning of this invention may include patients with advanced or late stage type 2 diabetes mellitus (including patients with failure to conventional antidiabetic therapy), such as e.g. patients with inadequate glycemic control on one, two or more conventional oral and/or non-oral antidiabetic drugs as defined herein, such as e.g. patients with insufficient glycemic control despite (mono-)therapy with metformin, a thiazolidinedione (particularly pioglitazone), a sulphonylurea, a glinide, GLP-1 or GLP-1 analogue, insulin or insulin analogue, or an α-glucosidase inhibitor, or despite dual combination therapy with metformin/sulphonylurea, metformin/thiazolidinedione (particularly pioglitazone), sulphonylurea/α-glucosidase inhibitor, pioglitazone/sulphonylurea, metformin/insulin, pioglitazone/insulin or sulphonylurea/insulin. Thus, in an embodiment, the therapies described herein may be used in patients experienced with therapy, e.g. with conventional oral and/or non-oral antidiabetic mono- or dual or triple combination medication as mentioned herein.

A further embodiment of diabetic patients within the meaning of this invention refers to patients ineligible for metformin therapy including
patients for whom metformin therapy is contraindicated, e.g. patients having one or more contraindications against metformin therapy according to label, such as for example patients with at least one contraindication selected from:
   renal disease, renal impairment or renal dysfunction (e.g., as specified by product information of locally approved metformin),
   dehydration,
   unstable or acute congestive heart failure,
   acute or chronic metabolic acidosis, and
   hereditary galactose intolerance;
      and
patients who suffer from one or more intolerable side effects attributed to metformin, particularly gastrointestinal side effects associated with metformin, such as for example patients suffering from at least one gastrointestinal side effect selected from:
   nausea,
   vomiting,
   diarrhea,
   intestinal gas, and
   severe abdominal discomfort.

A further embodiment of the diabetes patients which may be amenable to the therapies of this invention may include, without being limited, those diabetes patients for whom normal metformin therapy is not appropriate, such as e.g. those diabetes patients who need reduced dose metformin therapy due to reduced tolerability, intolerability or contraindication against metformin or due to (mildly) impaired/reduced renal function (including elderly patients, such as e.g. 60-65 years).

A further embodiment of patients (e.g. diabetic and/or obese) within the meaning of this invention refers to patients having renal disease, renal dysfunction, or insufficiency or impairment of renal function (including mild, moderate and severe renal impairment), e.g. as suggested by elevated serum creatinine levels (e.g. serum creatinine levels above the upper limit of normal for their age, e.g. ≧130-150 μmol/l, or ≧1.5 mg/dl (≧136 μmol/l) in men and ≧1.4 mg/dl (≧124 μmol/l) in women) or abnormal creatinine clearance (e.g. glomerular filtration rate (GFR)≦30-60 ml/min).

In this context, for more detailed example, mild renal impairment may be e.g. suggested by a creatinine clearance of 50-80 ml/min (approximately corresponding to serum creatine levels of ≦1.7 mg/dL in men and ≦1.5 mg/dL in women); moderate renal impairment may be e.g. suggested by a creatinine clearance of 30-50 ml/min (approximately corresponding to serum creatinine levels of >1.7 to ≦3.0 mg/dL in men and >1.5 to ≦2.5 mg/dL in women); and severe renal impairment may be e.g. suggested by a creatinine clearance of <30 ml/min (approximately corresponding to serum creatinine levels of >3.0 mg/dL in men and >2.5 mg/dL in women). Patients with end-stage renal disease require dialysis (e.g. hemodialysis or peritoneal dialysis).

For other more detailed example, patients with renal disease, renal dysfunction or renal impairment include patients with chronic renal insufficiency or impairment, which can be stratified according to glomerular filtration rate (GFR, ml/min/1.73 m²) into 5 disease stages: stage 1 characterized by normal GFR 90 plus either persistent albuminuria or known structural or hereditary renal disease; stage 2 characterized by mild reduction of GFR (GFR 60-89) describing mild renal impairment; stage 3 characterized by moderate reduction of GFR (GFR 30-59) describing moderate renal impairment; stage 4 characterized by severe reduction of GFR (GFR 15-29) describing severe renal impairment; and terminal stage 5 characterized by requiring dialysis or GFR <15 describing established kidney failure (end-stage renal disease, ESRD).

A further embodiment of patients (e.g. diabetic and/or obese) within the meaning of this invention refers to type 2 diabetes and/or obesity patients with or at risk of developing renal complications, such as diabetic nephropathy (including chronic and progressive renal insufficiency, albuminuria, proteinuria, fluid retention in the body (edema) and/or hypertension).

Examples of metabolic disorders or diseases amenable by the therapy of this invention may include, without being limited to, type 1 diabetes, type 2 diabetes, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, fasting or postprandial hyperlipidemia such as postprandial lipemia (e.g. postprandial hypertriglyceridemia), hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome.

The present invention further relates to a certain DPP-4 inhibitor (preferably linagliptin, optionally in combination with one or more other active agents, such as e.g. including a GLP-1 receptor agonist, particularly a short acting GLP-1, GLP-1 mimetic or GLP-1 analogue, such as exenatide or native GLP-1, and/or (preferably) an α-glucosidase inhibitor, such as voglibose) for use in at least one of the following methods:
preventing, slowing the progression of, delaying the onset of or treating a metabolic disorder or disease, such as e.g. type 1 diabetes mellitus, type 2 diabetes mellitus, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, postprandial lipemia (e.g. postprandial hypertriglyceridemia), hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome;
improving and/or maintaining glycemic control and/or for reducing of fasting plasma glucose, of postprandial plasma glucose, of postabsorptive plasma glucose and/or of glycosylated hemoglobin HbA1c, or preventing, reducing the risk of, slowing the progression of, delaying the onset of or treating worsening or deterioration of glycemic control, need for insulin therapy or elevated HbA1c despite treatment;
preventing, slowing, delaying the onset of or reversing progression from pre-diabetes, impaired glucose tolerance (IGT), impaired fasting blood glucose (IFG), insulin resistance and/or from metabolic syndrome to type 2 diabetes mellitus;
preventing, reducing the risk of, slowing the progression of, delaying the onset of or treating of complications of diabetes mellitus such as micro- and macrovascular diseases, such as nephropathy, micro- or macroalbuminuria, proteinuria, retinopathy, cataracts, neuropathy, learning or memory impairment, neurodegenerative or cognitive disorders, cardio- or cerebrovascular diseases, tissue ischaemia, diabetic foot or ulcus, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke;
reducing body weight and/or body fat and/or liver fat and/or intra-myocellular fat or preventing an increase in body weight and/or body fat and/or liver fat and/or intra-myocellular fat or facilitating a reduction in body weight and/or body fat and/or liver fat and/or intra-myocellular fat;
preventing, slowing, delaying the onset of or treating the degeneration of pancreatic beta cells and/or the decline of the functionality of pancreatic beta cells and/or for improving, preserving and/or restoring the functionality of pancreatic beta cells and/or stimulating and/or restoring or protecting the functionality of pancreatic insulin secretion;
preventing, slowing, delaying the onset of or treating non alcoholic fatty liver disease (NAFLD) including hepatic steatosis, non-alcoholic steatohepatitis (NASH) and/or liver fibrosis (such as e.g. preventing, slowing the progression, delaying the onset of, attenuating, treating or reversing hepatic steatosis, (hepatic) inflammation and/or an abnormal accumulation of liver fat);
preventing, slowing the progression of, delaying the onset of or treating type 2 diabetes with failure to conventional antidiabetic mono- or combination therapy;
achieving a reduction in the dose of conventional antidiabetic medication required for adequate therapeutic effect;
reducing the risk for adverse effects associated with conventional antidiabetic medication (e.g. hypoglycemia or weight gain); and/or
maintaining and/or improving the insulin sensitivity and/or for treating or preventing hyperinsulinemia and/or insulin resistance;
   in a patient in need thereof (such as e.g. a patient as described herein, for example a patient having overweight, obesity and/or diabetes),
   particularly in a patient who is further in need of reduction in, facilitation of a reduction in, or prevention of an increase in caloric intake, excessive food energy intake, body weight, total body fat mass, total body fat percentage, visceral fat and/or over eating, and/or
   in a patient who is further in need of modification of and/or reduction in intake of food, particularly wherein said food is rich in fat and/or in carbohydrate (e.g. high caloric, palatable, sweet and/or fatty food), for example such food which has a high glycemic index and/or
   wherein the amount of mono- and/or di-saccharides constitute a large portion of the total amount of carbohydrate and/or wherein a large portion of the total amount of energy stems from fat, and/or
   in a patient who is further in need of reduction in intake of high caloric (palatable, rich in fat and/or carbohydrate) food and/or of regulation or shift in food or taste preference or choice of food away from unhealthy food (e.g. as described herein) and towards healthy food (e.g. as described herein), such as in a patient in need of decrease in the amount of unhealthy and/or high caloric (e.g. palatable, rich in fat and/or carbohydrate) food intake and/or in need of increase in the amount of healthy and/or low caloric food intake.

In a particular embodiment, the present invention further relates to a method of:

reducing, facilitation of a reduction in, or preventing an increase in caloric intake, excessive food energy intake, body weight, total body fat mass, total body fat percentage, visceral fat and/or over eating, and/or
modifying and/or reducing intake of food, particularly wherein said food is rich in fat and/or in carbohydrate (e.g. high caloric, palatable, sweet and/or fatty food), for example such food which has a high glycemic index and/or wherein the amount of mono- and/or di-saccharides constitute a large portion of the total amount of carbohydrate and/or wherein a large portion of the total amount of energy stems from fat, and/or
reducing intake of high caloric (palatable, rich in fat and/or carbohydrate) food and/or regulating or shifting food or taste preference or choice of food away from unhealthy food (e.g. as described herein) and towards healthy food (e.g. as described herein), such as decreasing the amount of unhealthy and/or high caloric (e.g. palatable, rich in fat and/or carbohydrate) food intake and/or increasing the amount of healthy and/or low caloric food intake,
in a subject (such as e.g. a human subject in need thereof, for example a patient having overweight, obesity and/or diabetes),
said method comprising administering (such as e.g. simultaneously, e.g. twice daily, e.g. subcutaneously or transdermally) an effective amount of a certain DPP-4 inhibitor as defined herein (preferably linagliptin, such as e.g. in a subcutaneous amount of 0.3-10 mg or 0.1-30 mg, preferably from 1 to 5 mg or from 1 to 10 mg, e.g. 2.5 mg or 5 mg per day) and a GLP-1 receptor agonist, particularly a short acting GLP-1, GLP-1 mimetic or GLP-1 analogue, such as exenatide or native GLP-1, and/or an alpha-glucosidase inhibitor (e.g. voglibose, miglitol or acarbose, such as in low dose form, preferably voglibose) to the subject.

Within this invention it is to be understood that the combinations, compositions or combined uses according to this invention may envisage the simultaneous, sequential or separate administration of the active components or ingredients.

In this context, “combination” or “combined” within the meaning of this invention may include, without being limited, fixed and non-fixed (e.g. free) forms (including kits) and uses, such as e.g. the simultaneous, sequential or separate use of the components or ingredients.

The combined administration of this invention may take place by administering the active components or ingredients together, such as e.g. by administering them simultaneously in one single or in two separate formulations or dosage forms. Alternatively, the administration may take place by administering the active components or ingredients sequentially, such as e.g. successively in two separate formulations or dosage forms.

For the combination therapy of this invention the active components or ingredients may be administered separately (which implies that they are formulated separately) or formulated altogether (which implies that they are formulated in the same preparation or in the same dosage form). Hence, the administration of one element of the combination of the present invention may be prior to, concurrent to, or subsequent to the administration of the other element of the combination.

For example, in one embodiment, for the combination therapy according to this invention the DPP-4 inhibitor and the GLP-1 receptor agonist (particularly short acting GLP-1, GLP-1 mimetic or GLP-1 analogue, such as exenatide or native GLP-1) are administered in different formulations or different dosage forms.

In another embodiment, for the combination therapy according to this invention the DPP-4 inhibitor and the GLP-1 receptor agonist (particularly short acting GLP-1, GLP-1 mimetic or GLP-1 analogue, such as exenatide or native GLP-1) are administered in the same formulation or in the same dosage form.

In a further embodiment, for the combination therapy according to this invention the DPP-4 inhibitor and the GLP-1 receptor agonist (particularly short acting GLP-1, GLP-1 mimetic or GLP-1 analogue, such as exenatide or native GLP-1) are administered simultaneously.

In a further embodiment, for the combination therapy according to this invention the DPP-4 inhibitor is administered orally and the GLP-1 receptor agonist (particularly short acting GLP-1, GLP-1 mimetic or GLP-1 analogue, such as exenatide or native GLP-1) is administered subcutaneously.

In a further embodiment, for the combination therapy according to this invention the DPP-4 inhibitor and the GLP-1 receptor agonist (particularly short acting GLP-1, GLP-1 mimetic or GLP-1 analogue, such as exenatide or native GLP-1) are each administered subcutaneously.

In a further embodiment, for the combination therapy according to this invention the DPP-4 inhibitor and the GLP-1 receptor agonist (particularly short acting GLP-1, GLP-1 mimetic or GLP-1 analogue, such as exenatide or native GLP-1) are administered simultaneously and each subcutaneously.

The present invention also provides a kit-of-parts or combination therapeutic product comprising

a) a pharmaceutical composition comprising a DPP-4 inhibitor (preferably linagliptin) as defined herein, optionally together with one or more pharmaceutically acceptable carriers and/or diluents, and
b) a pharmaceutical composition comprising a GLP-1 receptor agonist (particularly short acting GLP-1, GLP-1 mimetic or GLP-1 analogue, such as exenatide or native GLP-1) as defined herein, optionally together with one or more pharmaceutically acceptable carriers and/or diluents.

The present invention also provides a kit comprising

a) a DPP-4 inhibitor (preferably linagliptin) as defined herein, and
b) a GLP-1 receptor agonist (particularly short acting GLP-1, GLP-1 mimetic or GLP-1 analogue, such as exenatide or native GLP-1) as defined herein,
and, optionally, instructions directing use of the DPP-4 inhibitor and the GLP-1 receptor agonist in combination (e.g. simultaneously), e.g. for a purpose of this invention.

The present invention also provides a pharmaceutical composition or fixed dose combination comprising

a) a DPP-4 inhibitor (preferably linagliptin) as defined herein, and
b) a GLP-1 receptor agonist (particularly short acting GLP-1, GLP-1 mimetic or GLP-1 analogue, such as exenatide or native GLP-1) as defined herein;
and, optionally, one or more pharmaceutically acceptable carriers and/or diluents.

The present invention also provides a transdermal or subcutaneous (injectable) pharmaceutical composition, delivery system or device for systemic use comprising

a) a DPP-4 inhibitor (preferably linagliptin) as defined herein, and, optionally,
b) a GLP-1 receptor agonist (particularly short acting GLP-1, GLP-1 mimetic or GLP-1 analogue, such as exenatide or native GLP-1) as defined herein;
and, optionally, one or more pharmaceutically acceptable carriers and/or diluents.

Further, the present invention relates to a pharmaceutical composition according to this invention comprising

a DPP-4 inhibitor (preferably linagliptin) as defined herein, and, optionally,
a GLP-1 receptor agonist (particularly short acting GLP-1, GLP-1 mimetic or GLP-1 analogue, such as exenatide or native GLP-1) as defined herein;
and, optionally, one or more pharmaceutically acceptable carriers and/or diluents, said composition being for subcutaneous administration to the patient in need thereof, e.g. by injection.

Unless otherwise noted, combination therapy may refer to first line, second line or third line therapy, or initial or add-on combination therapy or replacement therapy.

With respect to embodiment A, the methods of synthesis for the DPP-4 inhibitors according to embodiment A of this invention are known to the skilled person. Advantageously, the DPP-4 inhibitors according to embodiment A of this invention can be prepared using synthetic methods as described in the literature. Thus, for example, purine derivatives of formula (I) can be obtained as described in WO 2002/068420, WO 2004/018468, WO 2005/085246, WO 2006/029769 or WO 2006/048427, the disclosures of which are incorporated herein. Purine derivatives of formula (II) can be obtained as described, for example, in WO 2004/050658 or WO 2005/110999, the disclosures of which are incorporated herein.

Purine derivatives of formula (III) and (IV) can be obtained as described, for example, in WO 2006/068163, WO 2007/071738 or WO 2008/017670, the disclosures of which are incorporated herein. The preparation of those DPP-4 inhibitors, which are specifically mentioned hereinabove, is disclosed in the publications mentioned in connection therewith. Polymorphous crystal modifications and formulations of particular DPP-4 inhibitors are disclosed in WO 2007/128721 and WO 2007/128724, respectively, the disclosures of which are incorporated herein in their entireties. Formulations of particular DPP-4 inhibitors with metformin or other combination partners are described in WO 2009/121945, the disclosure of which is incorporated herein in its entirety.

Typical dosage strengths of the dual fixed combination (tablet) of linagliptin/metformin IR (immediate release) are 2.5/500 mg, 2.5/850 mg and 2.5/1000 mg, which may be administered 1-3 times a day, particularly twice a day.

Typical dosage strengths of the dual fixed combination (tablet) of linagliptin/metformin XR (extended release) are 5/500 mg, 5/1000 mg and 5/1500 mg (each one tablet) or 2.5/500 mg, 2.5/750 mg and 2.5/1000 mg (each two tablets), which may be administered 1-2 times a day, particularly once a day, preferably to be taken in the evening with meal.

The present invention further provides a DPP-4 inhibitor as defined herein for use in (add-on or initial) combination therapy with metformin (e.g. in a total daily amount from 500 to 2000 mg metformin hydrochloride, such as e.g. 500 mg, 850 mg or 1000 mg once or twice daily).

With respect to embodiment B, the methods of synthesis for the DPP-4 inhibitors of embodiment B are described in the scientific literature and/or in published patent documents, particularly in those cited herein.

The elements of the combination of this invention may be administered by various ways, for example by oral, buccal, sublingual, enterical, parenteral (e.g., transdermal, intramuscular or subcutaneous), inhalative (e.g., liquid or powder inhalation, aerosol), pulmonary, intranasal (e.g. spray), intraperitoneal, vaginal, rectal, or topical routes of administration and may be formulated, alone or together, in suitable dosage unit formulations containing conventional non-toxic pharmaceutically acceptable carriers, adjuvants and vehicles appropriate for each route of administration.

In an embodiment, the DPP-4 inhibitor according to the invention is preferably administered orally. In another embodiment, the GLP-1 receptor agonist (particularly short acting GLP-1, GLP-1 mimetic or GLP-1 analogue, such as exenatide or native GLP-1) is preferably administered by injection (preferably subcutaneously).

Suitable doses and dosage forms of the DPP-4 inhibitors may be determined by a person skilled in the art and may include those described herein or in the relevant references.

For pharmaceutical application in warm-blooded vertebrates, particularly humans, the compounds of this invention are usually used in dosages from 0.001 to 100 mg/kg body weight, preferably at 0.01-15 mg/kg or 0.1-15 mg/kg, in each case 1 to 4 times a day. For this purpose, the compounds, optionally combined with other active substances, may be incorporated together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof into conventional galenic preparations such as plain or coated tablets, capsules, powders, suspensions or suppositories.

The pharmaceutical compositions according to this invention comprising the DPP-4 inhibitors as defined herein are thus prepared by the skilled person using pharmaceutically acceptable formulation excipients as described in the art and appropriate for the desired route of administration. Examples of such excipients include, without being restricted to diluents, binders, carriers, fillers, lubricants, flow promoters, crystallisation retardants, disintegrants, solubilizers, colorants, pH regulators, surfactants and emulsifiers.

Oral formulations or dosage forms of the DPP-4 inhibitor of this invention may be prepared according to known techniques.

Examples of suitable diluents for compounds according to embodiment A include cellulose powder, calcium hydrogen phosphate, erythritol, low substituted hydroxypropyl cellulose, mannitol, pregelatinized starch or xylitol.

A pharmaceutical composition or dosage form (e.g. oral tablet) of a DPP-4 inhibitor according to embodiment A of the invention may typically contain as excipients (in addition to an active ingredient), for example: one or more diluents, a binder, a disintegrant, and a lubricant, preferably each as disclosed herein-below. In an embodiment, the disintegrant may be optional.

Examples of suitable lubricants for compounds according to embodiment A include talc, polyethyleneglycol, calcium behenate, calcium stearate, hydrogenated castor oil or magnesium stearate.

Examples of suitable binders for compounds according to embodiment A include copovidone (copolymerisates of vinylpyrrolidon with other vinylderivates), hydroxypropyl methylcellulose (HPMC), hydroxypropylcellulose (HPC), polyvinylpyrrolidon (povidone), pregelatinized starch, or low-substituted hydroxypropylcellulose (L-HPC).

Examples of suitable disintegrants for compounds according to embodiment A include corn starch or crospovidone.

Suitable methods of preparing (oral) preparations or dosage forms of the DPP-4 inhibitors according to embodiment A of the invention are
direct tabletting of the active substance in powder mixtures with suitable tabletting excipients;
granulation with suitable excipients and subsequent mixing with suitable excipients and subsequent tabletting as well as film coating; or
packing of powder mixtures or granules into capsules.

Suitable granulation methods are
wet granulation in the intensive mixer followed by fluidised bed drying;
one-pot granulation;
fluidised bed granulation; or
dry granulation (e.g. by roller compaction) with suitable excipients and subsequent tabletting or packing into capsules.

An exemplary composition (e.g. tablet core) of a DPP-4 inhibitor according to embodiment A of the invention comprises the first diluent mannitol, pregelatinized starch as a second diluent with additional binder properties, the binder copovidone, the disintegrant corn starch, and magnesium stearate as lubricant; wherein copovidone and/or corn starch may be optional.

A tablet of a DPP-4 inhibitor according to embodiment A of the invention may be film coated, preferably the film coat comprises hydroxypropylmethylcellulose (HPMC), polyethylene glycol (PEG), talc, titanium dioxide and iron oxide (e.g. red and/or yellow).

In a further embodiment, the DPP-4 inhibitor according to the invention is preferably administered by injection (preferably subcutaneously). In another embodiment, the GLP-1 receptor agonist (particularly short acting GLP-1, GLP-1 mimetic or GLP-1 analogue, such as exenatide or native GLP-1) is preferably administered by injection (preferably subcutaneously) as well.

Injectable formulations of the GLP-1 receptor agonist (particularly short acting GLP-1, GLP-1 mimetic or GLP-1 analogue, such as exenatide or native GLP-1) and/or the DPP-4 inhibitor of this invention (particularly for subcutaneous use) may be prepared according to known formulation techniques, e.g. using suitable liquid carriers, which usually comprise sterile water, and, optionally, further additives such as e.g. preservatives, pH adjusting agents, buffering agents, isotoning agents, solubility aids and/or tensides or the like, to obtain injectable solutions or suspensions. In addition, injectable formulations may comprise further additives, for example salts, solubility modifying agents or precipitating agents which retard release of the drug(s). In further addition, injectable GLP-1 formulations may comprise GLP-1 stabilizing agents (e.g. a surfactant).

For example, an injectable formulation (particularly for subcutaneous use) containing the GLP-1 receptor agonist (e.g. exenatide), optionally together with the DPP-4 inhibitor of this invention, may further comprise the following additives: a tonicity-adjusting agent (such as e.g. mannitol), an antimicrobial preservative (such as e.g. metacresol), a buffer or pH adjusting agent (such as e.g. glacial acetic acid and sodium acetate trihydrate in water for injection as a buffering solution at pH 4.5), and optionally a solubilizing and/or stabilizing agent (such as e.g. a surfactant or detergent).

In a further embodiment, the DPP-4 inhibitor according to the invention is preferably administered by a transdermal delivery system. In another embodiment, the GLP-1 receptor agonist (particularly short acting GLP-1, GLP-1 mimetic or GLP-1 analogue, such as exenatide or native GLP-1) is preferably administered by a transdermal delivery system as well.

Transdermal formulations (e.g. for transdermal patches or gels) of the GLP-1 receptor agonist (particularly short acting GLP-1, GLP-1 mimetic or GLP-1 analogue, such as exenatide or native GLP-1) and/or the DPP-4 inhibitor of this invention may be prepared according to known formulation techniques, e.g. using suitable carriers and, optionally, further additives. To facilitate transdermal passage, different methodologies and systems may be used, such as e.g. techniques involving formation of microchannels or micropores in the skin, such as e.g. iontophoresis (based on low-level electrical current), sonophoresis (based on low-frequency ultrasound) or microneedling, or the use of drug-carrier agents (e.g. elastic or lipid vesicles such as transfersomes) or permeation enhancers.

For further details on dosage forms, formulations and administration of DPP-4 inhibitors of this invention and/or GLP-1 receptor agonist of this invention, reference is made to scientific literature and/or published patent documents, particularly to those cited herein.

The pharmaceutical compositions (or formulations) may be packaged in a variety of ways. Generally, an article for distribution includes one or more containers that contain the one or more pharmaceutical compositions in an appropriate form. Tablets are typically packed in an appropriate primary package for easy handling, distribution and storage and for assurance of proper stability of the composition at prolonged contact with the environment during storage. Primary containers for tablets may be bottles or blister packs.

A suitable bottle, e.g. for a pharmaceutical composition or combination (tablet) comprising a DPP-4 inhibitor according to embodiment A of the invention, may be made from glass or polymer (preferably polypropylene (PP) or high density polyethylene (HD-PE)) and sealed with a screw cap. The screw cap may be provided with a child resistant safety closure (e.g. press-and-twist closure) for preventing or hampering access to the contents by children. If required (e.g. in regions with high humidity), by the additional use of a desiccant (such as e.g. bentonite clay, molecular sieves, or, preferably, silica gel) the shelf life of the packaged composition can be prolonged.

A suitable blister pack, e.g. for a pharmaceutical composition or combination (tablet) comprising a DPP-4 inhibitor according to embodiment A of the invention, comprises or is formed of a top foil (which is breachable by the tablets) and a bottom part (which contains pockets for the tablets). The top foil may contain a metallic foil, particularly aluminium or aluminium alloy foil (e.g. having a thickness of 20 μm to 45 μm, preferably 20 μm to 25 μm) that is coated with a heat-sealing polymer layer on its inner side (sealing side). The bottom part may contain a multi-layer polymer foil (such as e.g. poly(vinyl chloride) (PVC) coated with poly(vinylidene choride) (PVDC); or a PVC foil laminated with poly(chlorotriflouroethylene) (PCTFE)) or a multi-layer polymer-metal-polymer foil (such as e.g. a cold-formable laminated PVC/aluminium/polyamide composition).

To ensure a long storage period especially under hot and wet climate conditions an additional overwrap or pouch made of a multi-layer polymer-metal-polymer foil (e.g. a laminated polyethylene/aluminium/polyester composition) may be used for the blister packs. Supplementary desiccant (such as e.g. bentonite clay, molecular sieves, or, preferably, silica gel) in this pouch package may prolong the shelf life even more under such harsh conditions.

Solutions for injection may be available in typical suitable presentation forms such as vials, cartridges or prefilled (disposable) pens, which may be further packaged.

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

With respect to the first embodiment (embodiment A), the dosage typically required of the DPP-4 inhibitors mentioned herein in embodiment A when administered intravenously is 0.1 mg to 10 mg, preferably 0.25 mg to 5 mg, and when administered orally is 0.5 mg to 100 mg, preferably 2.5 mg to 50 mg or 0.5 mg to 10 mg, more preferably 2.5 mg to 10 mg or 1 mg to 5 mg, in each case 1 to 4 times a day. Thus, e.g. the dosage of 1-[(4-methyl-quinazolin-2-yl)methyl]-3-methyl-7-(2-butyn-1-yl)-8-(3-(R)-amino-piperidin-1-yl)-xanthine when administered orally is 0.5 mg to 10 mg per patient per day, preferably 2.5 mg to 10 mg or 1 mg to 5 mg per patient per day.

For example, doses of linagliptin when administered subcutaneously or i.v. for human patients are in the range of 0.3-10 mg, preferably from 1 to 5 mg, particularly 2.5 mg, per patient per day.

In a further embodiment, for example, doses of linagliptin when administered subcutaneously for human patients (such as e.g. in obese human patients or for treating obesity) are in the range of 0.1-30 mg, preferably from 1 to 10 mg, particularly 5 mg, per patient per day.

A dosage form prepared with a pharmaceutical composition comprising a DPP-4 inhibitor mentioned herein in embodiment A contain the active ingredient in a dosage range of 0.1-100 mg. Thus, e.g. particular oral dosage strengths of 1-[(4-methyl-quinazolin-2-yl)methyl]-3-methyl-7-(2-butyn-1-yl)-8-(3-(R)-amino-piperidin-1-yl)-xanthine are 0.5 mg, 1 mg, 2.5 mg, 5 mg and 10 mg.

With respect to the second embodiment (embodiment B), the doses of DPP-4 inhibitors mentioned herein in embodiment B to be administered to mammals, for example human beings, of, for example, approximately 70 kg body weight, may be generally from about 0.5 mg to about 350 mg, for example from about 10 mg to about 250 mg, preferably 20-200 mg, more preferably 20-100 mg, of the active moiety per person per day, or from about 0.5 mg to about 20 mg, preferably 2.5-10 mg, per person per day, divided preferably into 1 to 4 single doses which may, for example, be of the same size. Single oral dosage strengths comprise, for example, 10, 25, 40, 50, 75, 100, 150 and 200 mg of the DPP-4 inhibitor active moiety.

An oral dosage strength of the DPP-4 inhibitor sitagliptin is usually between 25 and 200 mg of the active moiety. A recommended dose of sitagliptin is 100 mg calculated for the active moiety (free base anhydrate) once daily. Unit dosage strengths of sitagliptin free base anhydrate (active moiety) are 25, 50, 75, 100, 150 and 200 mg. Particular unit dosage strengths of sitagliptin (e.g. per tablet) are 25, 50 and 100 mg. An equivalent amount of sitagliptin phosphate monohydrate to the sitagliptin free base anhydrate is used in the pharmaceutical compositions, namely, 32.13, 64.25, 96.38, 128.5, 192.75, and 257 mg, respectively. Adjusted dosages of 25 and 50 mg sitagliptin are used for patients with renal failure. Typical dosage strengths of the dual combination of sitagliptin/metformin are 50/500 mg and 50/1000 mg.

An oral dosage range of the DPP-4 inhibitor vildagliptin is usually between 10 and 150 mg daily, in particular between 25 and 150 mg, 25 and 100 mg or 25 and 50 mg or 50 and 100 mg daily. Particular examples of daily oral dosage are 25, 30, 35, 45, 50, 55, 60, 80, 100 or 150 mg. In a more particular aspect, the daily administration of vildagliptin may be between 25 and 150 mg or between 50 and 100 mg. In another more particular aspect, the daily administration of vildagliptin may be 50 or 100 mg. The application of the active ingredient may occur up to three times a day, preferably one or two times a day. Particular dosage strengths are 50 mg or 100 mg vildagliptin. Typical dosage strengths of the dual combination of vildagliptin/metformin are 50/850 mg and 50/1000 mg.

Alogliptin may be administered to a patient at an oral daily dose of between 5 mg/day and 250 mg/day, optionally between 10 mg and 200 mg, optionally between 10 mg and 150 mg, and optionally between 10 mg and 100 mg of alogliptin (in each instance based on the molecular weight of the free base form of alogliptin). Thus, specific oral dosage amounts that may be used include, but are not limited to 10 mg, 12.5 mg, 20 mg, 25 mg, 50 mg, 75 mg and 100 mg of alogliptin per day. Alogliptin may be administered in its free base form or as a pharmaceutically acceptable salt.

Saxagliptin may be administered to a patient at an oral daily dose of between 2.5 mg/day and 100 mg/day, optionally between 2.5 mg and 50 mg. Specific oral dosage amounts that may be used include, but are not limited to 2.5 mg, 5 mg, 10 mg, 15 mg, 20 mg, 30 mg, 40 mg, 50 mg and 100 mg of saxagliptin per day. Typical dosage strengths of the dual combination of saxagliptin/metformin are 2.5/500 mg and 2.5/1000 mg.

A special embodiment of the DPP-4 inhibitors of this invention refers to those orally administered DPP-4 inhibitors which are therapeutically efficacious at low dose levels, e.g. at oral dose levels <100 mg or <70 mg per patient per day, preferably <50 mg, more preferably <30 mg or <20 mg, even more preferably from 1 mg to 10 mg, particularly from 1 mg to 5 mg (more particularly 5 mg), per patient per day (if required, divided into 1 to 4 single doses, particularly 1 or 2 single doses, which may be of the same size, preferentially, administered orally once- or twice daily (more preferentially once-daily), advantageously, administered at any time of day, with or without food. Thus, for example, the daily oral amount 5 mg BI 1356 can be given in an once daily dosing regimen (i.e. 5 mg BI 1356 once daily) or in a twice daily dosing regimen (i.e. 2.5 mg BI 1356 twice daily), at any time of day, with or without food.

The GLP-1 receptor agonist (particularly short acting GLP-1, GLP-1 mimetic or GLP-1 analogue, such as exenatide or native GLP-1) are typically administered by subcutaneous injection, such as e.g. in an amount of 1-30 μg, 1-20 μg or 5-10 μg, e.g. once, twice or thrice daily. An embodiment thereof refers to those short-acting GLP-1 analogues (or any short-acting GLP-1 receptor agonists in general) that are to be administered at least twice daily, such as e.g. exenatide.

For example, exenatide is typically administered twice daily by subcutaneous injection (e.g. formulated as Byetta, e.g. in doses of 5-30 μg, particularly 5-20 μg, preferably 5-10 μg, specific dosage strengths are 5 or 10 μg).

The dosage of the active ingredients in the combinations and compositions in accordance with the present invention may be varied, although the amount of the active ingredients shall be such that a suitable dosage form is obtained. Hence, the selected dosage and the selected dosage form shall depend on the desired therapeutic effect, the route of administration and the duration of the treatment. Dosage ranges for the combination may be from the maximal tolerated dose for the single agent to lower doses, e.g. to one tenth of the maximal tolerated dose.

A particularly preferred DPP-4 inhibitor to be emphasized within the meaning of this invention is 1-[(4-methyl-quinazolin-2-yl)methyl]-3-methyl-7-(2-butyn-1-yl)-8-(3-(R)-amino-piperidin-1-yl)-xanthine (also known as BI 1356 or linagliptin). BI 1356 exhibits high potency, 24 h duration of action, and a wide therapeutic window. In patients with type 2 diabetes receiving multiple oral doses of 1, 2.5, 5 or 10 mg of BI 1356 once daily for 12 days, BI 1356 shows favourable pharmacodynamic and pharmacokinetic profile (see e.g. Table 3 below) with rapid attainment of steady state (e.g. reaching steady state plasma levels (>90% of the pre-dose plasma concentration on Day 13) between second and fifth day of treatment in all dose groups), little accumulation (e.g. with a mean accumulation ratio R_{A,AUC}≦1.4 with doses above 1 mg) and preserving a long-lasting effect on DPP-4 inhibition (e.g. with almost complete (>90%) DPP-4 inhibition at the 5 mg and 10 mg dose levels, i.e. 92.3 and 97.3% inhibition at steady state, respectively, and >80% inhibition over a 24 h interval after drug intake), as well as significant decrease in 2 h postprandial blood glucose excursions by ≧80% (already on Day 1) in doses ≧2.5 mg, and with the cumulative amount of unchanged parent compound excreted in urine on Day 1 being below 1% of the administered dose and increasing to not more than about 3-6% on Day 12 (renal clearance C_{LR,ss} is from about 14 to about 70 mL/min for the administered oral doses, e.g. for the 5 mg dose renal clearance is about 70 ml/min). In people with type 2 diabetes BI 1356 shows a placebo-like safety and tolerability. With low doses of about 5 mg, BI 1356 acts as a true once-daily oral drug with a full 24 h duration of DPP-4 inhibition. At therapeutic oral dose levels, BI 1356 is mainly excreted via the liver and only to a minor extent (about <7% of the administered oral dose) via the kidney. BI 1356 is primarily excreted unchanged via the bile. The fraction of BI 1356 eliminated via the kidneys increases only very slightly over time and with increasing dose, so that there will likely be no need to modify the dose of BI 1356 based on the patients' renal function. The non-renal elimination of BI 1356 in combination with its low accumulation potential and broad safety margin may be of significant benefit in a patient population that has a high prevalence of renal insufficiency and diabetic nephropathy.

TABLE 3
Geometric mean (gMean) and geometric coefficient of variation (gCV) of
pharmacokinetic parameters of BI 1356 at steady state (Day 12)

| | 1 mg | | 2.5 mg | | 5 mg | | 10 mg | |
|---|---|---|---|---|---|---|---|---|
| Parameter | gMean (gCV) | | gMean (gCV) | | gMean (gCV) | | gMean (gCV) | |
| AUC₀₋₂₄ | 40.2 | (39.7) | 85.3 | (22.7) | 118 | (16.0) | 161 | (15.7) |
| [nmol · h/L] | | | | | | | | |
| AUC_{T,ss} | 81.7 | (28.3) | 117 | (16.3) | 158 | (10.1) | 190 | (17.4) |
| [nmol · h/L] | | | | | | | | |
| Cₘₐₓ [nmol/L] | 3.13 | (43.2) | 5.25 | (24.5) | 8.32 | (42.4) | 9.69 | (29.8) |
| C_{max,ss} | 4.53 | (29.0) | 6.58 | (23.0) | 11.1 | (21.7) | 13.6 | (29.6) |
| [nmol/L] | | | | | | | | |
| tₘₐₓ* [h] | 1.50 | [1.00-3.00] | 2.00 | [1.00-3.00] | 1.75 | [0.92-6.02] | 2.00 | [1.50-6.00] |
| t_{max,ss}* [h] | 1.48 | [1.00-3.00] | 1.42 | [1.00-3.00] | 1.53 | [1.00-3.00] | 1.34 | [0.50-3.00] |
| T_{1/2,ss} [h] | 121 | (21.3) | 113 | (10.2) | 131 | (17.4) | 130 | (11.7) |
| Accumulation | 23.9 | (44.0) | 12.5 | (18.2) | 11.4 | (37.4) | 8.59 | (81.2) |
| t_{1/2,} [h] | | | | | | | | |
| R_{A,Cmax} | 1.44 | (25.6) | 1.25 | (10.6) | 1.33 | (30.0) | 1.40 | (47.7) |
| R_{A,AUC} | 2.03 | (30.7) | 1.37 | (8.2) | 1.33 | (15.0) | 1.18 | (23.4) |
| fe₀₋₂₄ [%] | NC | | 0.139 | (51.2) | 0.453 | (125) | 0.919 | (115) |
| fe_{T,ss} [%] | 3.34 | (38.3) | 3.06 | (45.1) | 6.27 | (42.2) | 3.22 | (34.2) |
| CL_{R,ss} | 14.0 | (24.2) | 23.1 | (39.3) | 70 | (35.0) | 59.5 | (22.5) |
| [mL/min] | | | | | | | | |

*median and range [min-max]
NC not calculated as most values below lower limit of quantification

As different metabolic functional disorders often occur simultaneously, it is quite often indicated to combine a number of different active principles with one another. Thus, depending on the functional disorders diagnosed, improved treatment outcomes may be obtained if a DPP-4 inhibitor is combined with one or more active substances which may be customary for the respective disorders, such as e.g. one or more active substances selected from among the other antidiabetic substances, especially active substances that lower the blood sugar level or the lipid level in the blood, raise the HDL level in the blood, lower blood pressure or are indicated in the treatment of atherosclerosis or obesity.

The DPP-4 inhibitors mentioned above—besides their use in mono-therapy—may also be used in conjunction with other active substances, by means of which improved treatment results can be obtained. Such a combined treatment may be given as a free combination of the substances or in the form of a fixed combination, for example in a tablet or capsule. Pharmaceutical formulations of the combination partner needed for this may either be obtained commercially as pharmaceutical compositions or may be formulated by the skilled man using conventional methods. The active substances which may be obtained commercially as pharmaceutical compositions are described in numerous places in the prior art, for example in the list of drugs that appears annually, the “Rote Liste®” of the federal association of the pharmaceutical industry, or in the annually updated compilation of manufacturers' information on prescription drugs known as the “Physicians' Desk Reference”.

Examples of antidiabetic combination partners are metformin; sulphonylureas such as glibenclamide, tolbutamide, glimepiride, glipizide, gliquidon, glibornuride and gliclazide; nateglinide; repaglinide; mitiglinide; thiazolidinediones such as rosiglitazone and pioglitazone; PPAR gamma modulators such as metaglidases; PPAR-gamma agonists such as e.g. rivoglitazone, mitoglitazone, INT-131 and balaglitazone; PPAR-gamma antagonists; PPAR-gamma/alpha modulators such as tesaglitazar, muraglitazar, aleglitazar, indeglitazar and KRP297; PPAR-gamma/alpha/delta modulators such as e.g. lobeglitazone; AMPK-activators such as AICAR; acetyl-CoA carboxylase (ACC1 and ACC2) inhibitors; diacylglycerol-acetyltransferase (DGAT) inhibitors; pancreatic beta cell GCRP agonists such as GPR119 agonists (SMT3-receptor-agonists), such as the GPR119 agonists 5-ethyl-2-{4-[4-(4-tetrazol-1-yl-phenoxymethyl)-thiazol-2-yl]-piperidin-1-yl}-pyrimidine or 5-[1-(3-isopropyl-[1,2,4]oxadiazol-5-yl)-piperidin-4-ylmethoxy]-2-(4-methanesulfonyl-phenyl)-pyridine; 11β-HSD-inhibitors; FGF19 agonists or analogues; alpha-glucosidase blockers such as acarbose, voglibose and miglitol; alpha2-antagonists; insulin and insulin analogues such as human insulin, insulin lispro, insulin glusilin, r-DNA-insulinaspart, NPH insulin, insulin detemir, insulin degludec, insulin tregopil, insulin zinc suspension and insulin glargin; Gastric inhibitory Peptide (GIP); amylin and amylin analogues (e.g. pramlintide or davalintide); GLP-1 and GLP-1 analogues such as Exendin-4, e.g. exenatide, exenatide LAR, liraglutide, taspoglutide, lixisenatide (AVE-0010), LY-2428757 (a PEGylated version of GLP-1), dulaglutide (LY-2189265), semaglutide or albiglutide; SGLT2-inhibitors such as e.g. dapagliflozin, sergliflozin (KGT-1251), atigliflozin, canagliflozin, ipragliflozin, luseogliflozin or tofogliflozin; inhibitors of protein tyrosine-phosphatase (e.g. trodusquemine); inhibitors of glucose-6-phosphatase; fructose-1,6-bisphosphatase modulators; glycogen phosphorylase modulators; glucagon receptor antagonists; phosphoenolpyruvatecarboxykinase (PEPCK) inhibitors; pyruvate dehydrogenasekinase (PDK) inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976) or of serine/threonine kinases; glucokinase/regulatory protein modulators incl. glucokinase activators; glycogen synthase kinase inhibitors; inhibitors of the SH2-domain-containing inositol 5-phosphatase type 2 (SHIP2); IKK inhibitors such as high-dose salicylate; JNK1 inhibitors; protein kinase C-theta inhibitors; beta 3 agonists such as ritobegron, YM 178, solabegron, talibegron, N-5984, GRC-1087, rafabegron, FMP825; aldosereductase inhibitors such as AS 3201, zenarestat, fidarestat, epalrestat, ranirestat, NZ-314, CP-744809, and CT-112; SGLT-1 or SGLT-2 inhibitors; KV 1.3 channel inhibitors; GPR40 modulators such as e.g. [(3S)-6-({2′,6′-dimethyl-4′-[3-(methylsulfonyl)propoxy]biphenyl-3-yl}methoxy)-2,3-dihydro-1-benzofuran-3-yl]acetic acid; SCD-1 inhibitors; CCR-2 antagonists; dopamine receptor agonists (bromocriptine mesylate [Cycloset]); 4-(3-(2,6-dimethylbenzyloxy)phenyl)-4-oxobutanoic acid; sirtuin stimulants; and other DPP IV inhibitors.

Metformin is usually given in doses varying from about 500 mg to 2000 mg up to 2500 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or preferably 500 mg to 1000 mg once or twice a day or about 500 mg to 2000 mg once a day. Particular dosage strengths may be 250, 500, 625, 750, 850 and 1000 mg of metformin hydrochloride.

For children 10 to 16 years of age, the recommended starting dose of metformin is 500 mg given once daily. If this dose fails to produce adequate results, the dose may be increased to 500 mg twice daily. Further increases may be made in increments of 500 mg weekly to a maximum daily dose of 2000 mg, given in divided doses (e.g. 2 or 3 divided doses). Metformin may be administered with food to decrease nausea.

A dosage of pioglitazone is usually of about 1-10 mg, 15 mg, 30 mg, or 45 mg once a day.

Rosiglitazone is usually given in doses from 4 to 8 mg once (or divided twice) a day (typical dosage strengths are 2, 4 and 8 mg).

Glibenclamide (glyburide) is usually given in doses from 2.5-5 to 20 mg once (or divided twice) a day (typical dosage strengths are 1.25, 2.5 and 5 mg), or micronized glibenclamide in doses from 0.75-3 to 12 mg once (or divided twice) a day (typical dosage strengths are 1.5, 3, 4.5 and 6 mg).

Glipizide is usually given in doses from 2.5 to 10-20 mg once (or up to 40 mg divided twice) a day (typical dosage strengths are 5 and 10 mg), or extended-release glibenclamide in doses from 5 to 10 mg (up to 20 mg) once a day (typical dosage strengths are 2.5, 5 and 10 mg).

Glimepiride is usually given in doses from 1-2 to 4 mg (up to 8 mg) once a day (typical dosage strengths are 1, 2 and 4 mg).

A dual combination of glibenclamide/metformin is usually given in doses from 1.25/250 once daily to 10/1000 mg twice daily (typical dosage strengths are 1.25/250, 2.5/500 and 5/500 mg).

A dual combination of glipizide/metformin is usually given in doses from 2.5/250 to 10/1000 mg twice daily (typical dosage strengths are 2.5/250, 2.5/500 and 5/500 mg).

A dual combination of glimepiride/metformin is usually given in doses from 1/250 to 4/1000 mg twice daily.

A dual combination of rosiglitazone/glimepiride is usually given in doses from 4/1 once or twice daily to 4/2 mg twice daily (typical dosage strengths are 4/1, 4/2, 4/4, 8/2 and 8/4 mg).

A dual combination of pioglitazone/glimepiride is usually given in doses from 30/2 to 30/4 mg once daily (typical dosage strengths are 30/4 and 45/4 mg).

A dual combination of rosiglitazone/metformin is usually given in doses from 1/500 to 4/1000 mg twice daily (typical dosage strengths are 1/500, 2/500, 4/500, 2/1000 and 4/1000 mg).

A dual combination of pioglitazone/metformin is usually given in doses from 15/500 once or twice daily to 15/850 mg thrice daily (typical dosage strengths are 15/500 and 15/850 mg).

The non-sulphonylurea insulin secretagogue nateglinide is usually given in doses from 60 to 120 mg with meals (up to 360 mg/day, typical dosage strengths are 60 and 120 mg); repaglinide is usually given in doses from 0.5 to 4 mg with meals (up to 16 mg/day, typical dosage strengths are 0.5, 1 and 2 mg). A dual combination of repaglinide/metformin is available in dosage strengths of 1/500 and 2/850 mg.

Acarbose is usually given in doses from 25 to 100 mg with meals (usually 3 times a day). Miglitol is usually given in doses from 25 to 100 mg with meals (usually 3 times a day). Voglibose is usually given in doses from 0.2 to 0.3 mg with meals (usually 3 times a day).

Examples of combination partners that lower the lipid level in the blood are HMG-CoA-reductase inhibitors such as simvastatin, atorvastatin, lovastatin, fluvastatin, pravastatin, pitavastatin and rosuvastatin; fibrates such as bezafibrate, fenofibrate, clofibrate, gemfibrozil, etofibrate and etofyllinclofibrate; nicotinic acid and the derivatives thereof such as acipimox; PPAR-alpha agonists; PPAR-delta agonists such as e.g. {4-[(R)-2-ethoxy-3-(4-trifluoromethyl-phenoxy)-propylsulfanyl]-2-methyl-phenoxy}-acetic acid; inhibitors of acyl-coenzyme A:cholesterolacyltransferase (ACAT; EC 2.3.1.26) such as avasimibe; cholesterol resorption inhibitors such as ezetimib; substances that bind to bile acid, such as cholestyramine, colestipol and colesevelam; inhibitors of bile acid transport; HDL modulating active substances such as D4F, reverse D4F, LXR modulating active substances and FXR modulating active substances; CETP inhibitors such as torcetrapib, JTT-705 (dalcetrapib) or compound 12 from WO 2007/005572 (anacetrapib); LDL receptor modulators; MTP inhibitors (e.g. lomitapide); and ApoB100 antisense RNA.

A dosage of atorvastatin is usually from 1 mg to 40 mg or 10 mg to 80 mg once a day.

Examples of combination partners that lower blood pressure are beta-blockers such as atenolol, bisoprolol, celiprolol, metoprolol and carvedilol; diuretics such as hydrochlorothiazide, chlortalidon, xipamide, furosemide, piretanide, torasemide, spironolactone, eplerenone, amiloride and triamterene; calcium channel blockers such as amlodipine, nifedipine, nitrendipine, nisoldipine, nicardipine, felodipine, lacidipine, lercanipidine, manidipine, isradipine, nilvadipine, verapamil, gallopamil and diltiazem; ACE inhibitors such as ramipril, lisinopril, cilazapril, quinapril, captopril, enalapril, benazepril, perindopril, fosinopril and trandolapril; as well as angiotensin II receptor blockers (ARBs) such as telmisartan, candesartan, valsartan, losartan, irbesartan, olmesartan, azilsartan and eprosartan.

A dosage of telmisartan is usually from 20 mg to 320 mg or 40 mg to 160 mg per day.

Examples of combination partners which increase the HDL level in the blood are Cholesteryl Ester Transfer Protein (CETP) inhibitors; inhibitors of endothelial lipase; regulators of ABC1; LXRalpha antagonists; LXRbeta agonists; PPAR-delta agonists; LXRalpha/beta regulators, and substances that increase the expression and/or plasma concentration of apolipoprotein A-I.

Examples of combination partners for the treatment of obesity are sibutramine; tetrahydrolipstatin (orlistat); alizyme (cetilistat); dexfenfluramine; axokine; cannabinoid receptor 1 antagonists such as the CB1 antagonist rimonobant; MCH-1 receptor antagonists; MC4 receptor agonists; NPY5 as well as NPY2 antagonists (e.g. velneperit); beta3-AR agonists such as SB-418790 and AD-9677; 5HT2c receptor agonists such as APD 356 (lorcaserin); myostatin inhibitors; Acrp30 and adiponectin; steroyl CoA desaturase (SCD1) inhibitors; fatty acid synthase (FAS) inhibitors; CCK receptor agonists; Ghrelin receptor modulators; Pyy 3-36; orexin receptor antagonists; and tesofensine; as well as the dual combinations bupropion/naltrexone, bupropion/zonisamide, topiramate/phentermine and pramlintide/metreleptin.

Examples of combination partners for the treatment of atherosclerosis are phospholipase A2 inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976); oxLDL antibodies and oxLDL vaccines; apoA-1 Milano; ASA; and VCAM-1 inhibitors.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### Examples

### Reduction of Body Weight, Fat Mass and Alteration of Food Preference by Linagliptin Optionally in Combination with a GLP-1 Receptor Agonist

### Model for Obesity:

Male Sprague Dawley rats are from Taconic at 6 weeks of age. Throughout the study the rats have ad libitum access to a two choice diet—regular Altromin 1324 rodent chow (Brogaarden, Denmark) and a high fat paste (HFD) made from Chocolate spread (Nutella, Ferrero Italy), Peanutbutter and powdered regular Altromin 1324 rodent chow (Brogaarden, Denmark). The animals are kept on the diet for 23 weeks before experimentation. One week prior to first dose (week 28), rats are single-housed.

Day 0 is the first day of dosing. Animals are subjected to bi-daily treatment (saline s.c., linagliptin 0.5 mg/kg, native GLP-1 0.4 mg/ml and the combination thereof) and are dosed between 7:00-9:00 AM and 15:00-17:00 PM. Animals are dosed for 32 days. Rats are terminated on study day 33.

### Key Findings

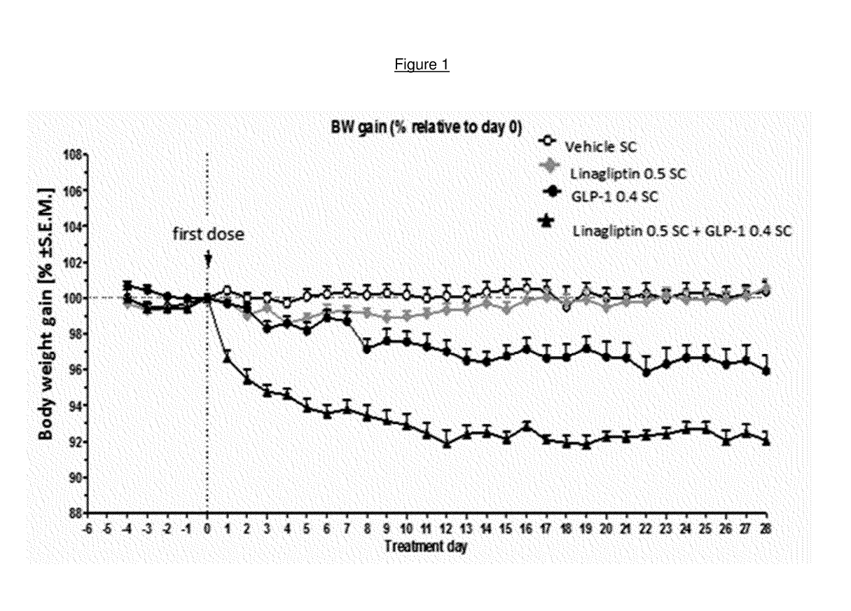

Bidaily s.c. treatment with linagliptin and native GLP-1 lead to a significant reduction of body weight (BW) up to 8%. Data show mean vaules+SEM of 10 animals (FIG. 1).

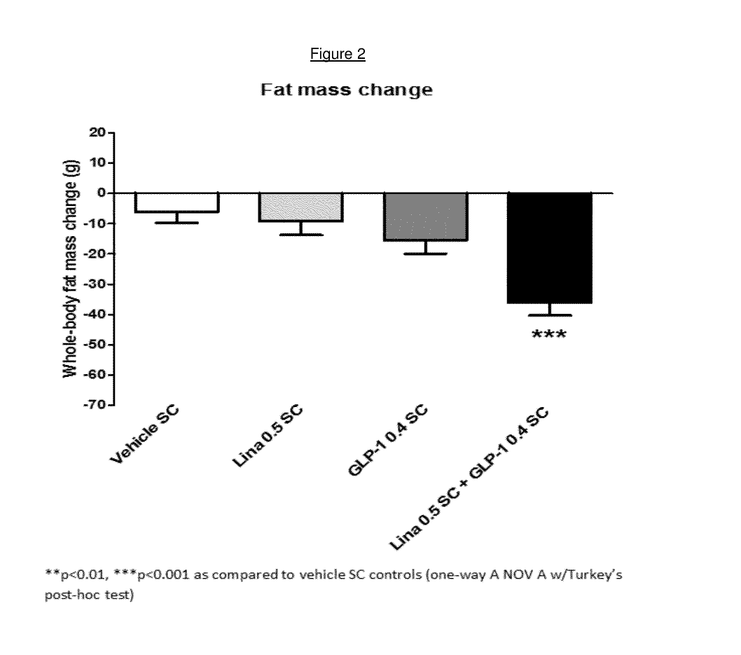

In accordance, fat mass is significantly decreased following application of the combination of linagliptin and native GLP-1 (FIG. 2).

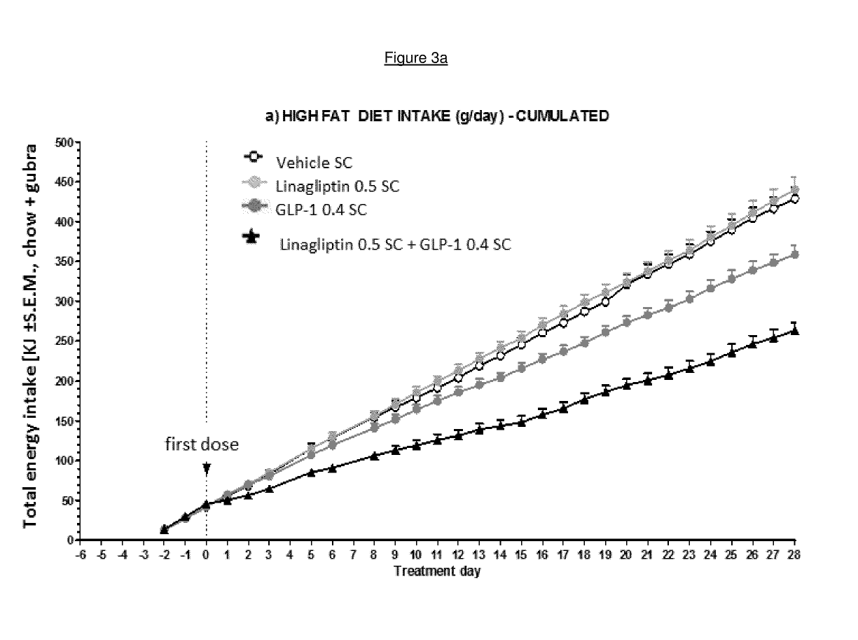

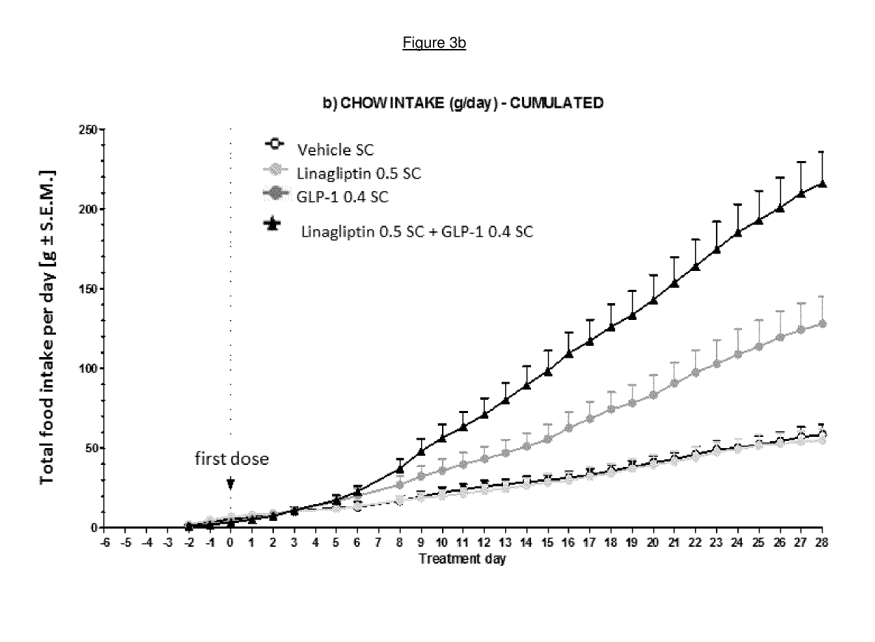

Linagliptin/GLP-1 treated animals possess altered food preferences in reduction intake of high fat diet a) and switching to chow diet b) (FIG. 3a, 3b).

### Co-Administration of the DPP-4 Inhibitor Linagliptin and Native GLP-1 Induce Body Weight Loss and Appetite Suppression

Linagliptin is a dipeptidyl peptidase (DPP)-4 inhibitor approved for the treatment of type 2 diabetes. DPP-4 inhibitors are weight-neutral, suggesting that elevation of endogenous incretin levels may not be sufficient to promote weight loss per se. Here it is evaluated the effect of subcutaneous co-administration of linagliptin and native GLP-1(7-36) in a rat model. In normal-weight rats, acute linagliptin treatment (0.5 mg/kg, sc BID) has no effect on nocturnal food intake, whereas GLP-1 treatment (0.4 mg/kg, sc BID) evokes a modest and short-lived suppression of food intake. In contrast, linagliptin and GLP-1 co-administration induces a robust acute anorectic response. In diet-induced obese (D10) rats, 14 days of linagliptin or GLP-1 monotherapy has no effect on body weight, whereas continuation with combined linagliptin (0.5 mg/kg, sc BID) and GLP-1 treatment (0.4 mg/kg, sc BID) for an additional 14 days induces a sustained decrease in food intake and body weight (−6.4±0.8%, compared with baseline body weight). Interestingly, the body weight-lowering effect of combined linagliptin and GLP-1 treatment is associated with a marked increase in chow preference at the expense of palatable high-fat-carbohydrate diet intake. In addition, combined linagliptin and GLP-1 treatment specifically increases preprodynorphin m RNA levels in the nucleus accumbens. These data demonstrate that combined treatment with linagliptin and GLP-1 synergistically reduces body weight in an obese rat model. This anti-obesity effect is caused by appetite suppression and change in diet preference, presumably associated with increased dynorphin activity in dopaminergic forebrain regions involved in reward anticipation and habit learning. In conclusion, linagliptin and GLP-1 (native GLP-1, GLP-1 receptor agonist, or GLP-1 mimetic or analogue, such as e.g. short acting GLP-1) co-administration may therefore hold promise as a novel therapeutic principle for combined weight and diabetes management as well as for appetite suppression, change in diet preference and body weight loss in obese patients.

### Glucose and Body Weight Control by Combination Linagliptin and Voglibose

It is investigated that combination therapy with the dipeptidyl peptidase-4 inhibitor linagliptin and the α-glucosidase inhibitor (AGI) voglibose can improve or maintain glycemic and body weight control. Male ZDF-Leprfa/Crl (diabetic fa/fa) rats fed standard chow are allocated (n=10/group) based on body weight and fasting plasma glucose (FPG). Rats are dosed (po) daily for 4 days in 2 studies—S1: vehicle, linagliptin (1 mg/kg), high-dose voglibose (10 mg/kg), or linagliptin+ voglibose; S2: as S1 except low-dose voglibose (1 mg/kg). An oral sucrose tolerance test (4 g/kg po) is performed on Day 4 and body weight is recorded daily. On Day 4, mean FPG levels are 7.39 and 8.18 mM, and mean insulin levels are 2.01 and 3.76 ng/mL, in S1 and S2, respectively. Improved glucose control is observed with linagliptin (S1-10%, S2-17%; both P<0.05), voglibose (S1-33%, P<0.001; S2-18%, P<0.01), linagliptin+ voglibose (S1-33%, S2-33%; both P<0.001) compared with vehicle. Improvement in glucose control is potentiated with linagliptin+ low-dose voglibose compared with either drug alone (P<0.01). Plasma active GLP-1 is increased 5 min after the sucrose load with linagliptin (S1 160%, P<0.01; S2 144%, P<0.001) and linagliptin+ voglibose (S1 834%, S2 639%; both P<0.001) compared with vehicle, and is larger than linagliptin or voglibose alone (P<0.001 for all). Compared with vehicle, linagliptin-induced improvements in glucose control are independent of changes in overall weight (S1+0.7%, S2-0.2%; both P=ns). In contrast, voglibose (S1-3.0%; S2-1.7%) and linagliptin+ voglibose (S1-3.4%; S2-2.0%) reduce overall weight compared with vehicle (P<0.001 for all). Therapy with linagliptin+ voglibose potentiates improvements in glucose control. This combination may minimize the side effects of AGIs because lower doses of voglibose may be required to maintain glycemic control, and may have additional beneficial effects due to the supra-additive increase in active GLP-1 levels.

What is claimed is:

1. A method of using a combination of a DPP-4 inhibitor, which is linagliptin, and an alpha-glucosidase inhibitor such as selected from voglibose, miglitol and acarbose, for one or more of the following:
treating and/or preventing a metabolic disease, and/or
improving body weight control, reducing body weight, inducing satiety, inhibiting gastric emptying, or reducing food intake, and/or
preventing, protecting against, reducing the likelihood or incidence of, or minimizing the side effects related to an alpha-glucosidase inhibitor,
optionally in combination with one or more other active agents,
in a patient in need thereof.

2. The method according to claim 1, wherein the side effects related to an alpha-glucosidase inhibitor are selected from gastrointestinal adverse effects such as dyspepsia, flatulence or diarrhea, or nausea or vomiting.

3. The method according to claim 1, wherein the patient is a diabetes patient, optionally having or being at-risk of micro- and/or macrovascular disease or complication.

4. The method of using according to claim 1, wherein the patient is a type 2 diabetes patient having or being at-risk of obesity or overweight, and/or having or being at-risk of renal impairment, and/or with indication on dose reduction of an alpha-glucosidase inhibitor.

5. The method according to claim 1, wherein the patient is a type 2 diabetes patient who is obese or overweight.

6. The method according to claim 1, wherein the patient is a type 2 diabetes patient in need of dose reduction of an alpha-glucosidase inhibitor.

7. The method according to claim 1, wherein the patient is a type 2 diabetes patient who has inadequate glycemic control despite diet and/or exercise alone as first line therapy.

8. The method according to claim 1, wherein the patient is a type 2 diabetes patient who has inadequate glycemic control despite mono therapy with an alpha-glucosidase inhibitor, in addition to diet and/or exercise as second line therapy.

9. The method according to claim 1, wherein the patient is a type 2 diabetes patient who has inadequate glycemic control despite combination therapy with an alpha-glucosidase inhibitor and one or more other antidiabetics, in addition to diet and/or exercise as third line therapy.

10. The method according to claim 1, wherein the alpha-glucosidase inhibitor is present or is used in low dose and/or less frequent dosing.

11. The method according to claim 1, wherein the alpha-glucosidase inhibitor is voglibose in a dose of 0.2-0.3 mg, each dose being administered one, two or three times a day, when co-administered with the DPP-4 inhibitor.

12. The method according to claim 1, wherein the alpha-glucosidase inhibitor is acarbose in la dose of 25-100 mg, each dose being administered one, two or three times a day, when co-administered with the DPP-4 inhibitor.

13. The method according to claim 1, wherein the alpha-glucosidase inhibitor is miglitol administered in a dose of such as 25-100 mg, each dose being administered one, two or three times a day, when co-administered with the DPP-4 inhibitor.

14. The method according to claim 1, wherein the DPP-4 inhibitor and the alpha-glucosidase inhibitor are administered simultaneously, sequentially or separately to the patient.

15. The method according to claim 1, wherein the DPP-4 inhibitor is 1-[(4-methyl-quinazolin-2-yl)methyl]-3-methyl-7-(2-butyn-1-yl)-8-(3-(R)-amino-piperidin-1-yl)-xanthine or a pharmaceutically acceptable salt thereof, and the metabolic disease is diabetes.
